SEQUENCE LISTING (80 sequences)

SEQ ID NO: 1 - DNA, 1515 bp, Artificial sequence
  Synthetic DNA
catcacagtcttgctaaagcgcaagcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagtttgcacgcctgccgttcgacgatatctctggaagatccgcgcgtaccgagttctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagctctagaaataattttgtttaactttaagaaggagatatacatatggctagcatgactggtggacagcaaatgggtcgcggatccatgtcacaacaacagcttgaatcaattatccagatgctcaagtcgcagcctatcgcgggcaaaccctcgattgcggaaacgcgcgcgggatttgagcagatggccgcgatgtttccggtcgaggctgacgtgaagagcgagccggtcaatgcgggcggcgtcaagtcagaatgggtgacgacgccgggcgcggactcgggccgcgcggtgctttatctgcacggtggcggctacgttatcggctcgatcaacacgcatcgcgcgttcgcgggacgaatctcgcgcgctgccaaagcgcgagtgctggtgatcgactaccggctcgcgcctgagcatccgtttcccgcagcggtggaggattccgtcgccgcatatcgctggatgctctcgactgggttgaagccctcgcgaatcgcagtcgcgggagactccgccggtggcggtctgacggttgcgacgctggttgcgattcgcgatgcgaagcttccagttccggccgcgggtgtggcgctgtcgccgtgggtcgatatggaaggagtcggcgattcgatgaagacgaaggcggctgtcgatccgatggtccagaaagacggcctgatcgagatggcaaaggcttatctcggcggcaaggatccgcgcacgccgctcgccgcgccgctctatgccgacctcgccggtctcccgccgctgctaatccaggtcggcaccgccgagactctgctcgacgactcgacgcggctggcggagcgcgcgcgcaaggcgggagtgaaggtaacgctggagccgtgggaaaacatggtccacgtcttccagatcttcgcgtcgattctcgacgaagggcagcaggcgatcgacaaaattggagcgttcatccgcgccaacgccgagtaa

SEQ ID NO: 2 - DNA, 741 bp, Escherichia coli
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaacacgcttgcattttgaaattcgttacaaggggaaatccgtaaacccgctgcgttatttgccgcagcgataaatcggcggaaccaggcttttgcttgaatgttccgtcaagggatcacgggtaggagccacc

SEQ ID NO: 3 - DNA, 1470 bp, Hirudo nipponia
atgaaagagatcgcggtgacaatagacgataagaacgttattgcctctgtctccgaaagtttccatggcgttgcctttgatgcgagtttattttcaccgaaagggttgtggtcatttgttgacatcacatcaccgaaattgtttaagctcttggagggtctctctccaggttatttccgcgtcggagggacgtttgctaactggttattctttgacctcgatgaaaacaacaaatggaaagactattgggcgtttaaagataagacacctgagacagcaacgatcacacgccggtggctttttcgaaaacagaacaacctgaagaaagaaacttttgatgatctggtcaaactcaccaaaggatctaaaatgcggctgctgtttgatttaaacgctgaagtcagaacaggttatgaaattggaaagaaaatgacatccacatgggatagcagtgaagctgaaaaactcttcaagtactgtgtctccaaaggctatggagataacattgactgggaacttggtaatgaaccggatcatacgtccgcacacaatcttactgaaaaacaagtaggagaggactttaaagccctgcataaagtgctggaaaaatatccgacgttgaataaaggatcattagtgggccctgacgttggctggatgggagtgtcttatgtgaaaggactggcggacggagccggggatcatgtaacggccttcacgcttcaccagtactactttgacggcaataccagcgatgtcagcacatacctggacgctacatatttcaaaaagctacagcagctttttgataaagtgaaggatgtccttaaaaatagcccacataaagataagccgctctggttaggagaaacctcttcaggctataattctggcaccaaagatgtttcggatcgttatgtaagcggttttcttactcttgacaaactgggtctgtctgccgcgaacaatgttaaggtagtaattagacaaacgatctataatggatattacggccttctggacaaaaacacacttgagccgaatccagattattggctgatgcatgtgcataactcgttagtagggaatacggtctttaaagtggacgttagtgatcctacaaataaagctagagtgtacgcacaatgcaccaaaacaaactcaaaacacactcagagtcgttactacaagggctcattaacgatctttgctttaaatgttggggatgaggatgtgacgttgaaaattgatcaatattctggcaagaagatttattcatatatacttacacctgaaggcggccaattgacttcacaaaaagttttattgaatggtaaagaattaaaacttgtcagcgatcagttgccggaactgaatgcagacgagtcgaagacctcgtttactctgtccccgaagacattcgggttctttgtagtcagcgatgcaaatgttgaagcgtgcaaaaagtaa

SEQ ID NO: 4 - DNA, 3666 bp, Artificial sequence
  Synthetic DNA
aactttaataaggagggatccataaaaggaggaaaatatatgagtaagtctgaaaatctttacagcgcagcgcgcgagctgatccctggcggtgtgaactcccctgttcgcgcctttactggcgtgggcggcactccactgtttatcgaaaaagcggacggcgcttatctgtacgatgttgatggcaaagcctatatcgattatgtcggttcctgggggccgatggtgctgggccataaccatccggcaatccgcaatgccgtgattgaagccgccgagcgtggtttaagctttggtgcaccaaccgaaatggaagtgaaaatggcgcaactggtgaccgaactggtcccgaccatggatatggtgcgcatggtgaactccggcactgaagcgaccatgagcgccatccgcctggcccgtggttttaccggtcgcgacaaaattattaaatttgaagggtgttaccatggtcacgctgactgcctgctggtgaaagccggttctggcgcactcacgttaggccagccaaactcgccgggcgttccggcagatttcgccaaatataccttaacctgtacttataatgatctggcttctgtacgcgccgcatttgagcaatacccgcaagagattgcctgtattatcgtcgagccggtggcaggcaatatgaactgtgttccgccgctgccagagttcctgccaggtctgcgcgcgctgtgcgacgaatttggcgcgttgctgatcatcgatgaagtgatgaccggtttccgcgtagcgctagctggcgcacaggattattacggcgtagtgccagatttaacctgcctcggcaaaatcatcggcggtggaatgccggtaggcgcattcggtggtcgtcgtgatgtaatggatgcgctggccccgacgggtccggtctatcaggcgggtacgctttccggtaacccgattgcgatggcagcgggtttcgcctgtctgaatgaagtcgcgcagccgggcgttcacgaaacgctggatgagctgacaacacgtctggcagaaggtctgctggaagcggcagaagaagccggaattccgctggtcgttaaccacgttggcggcatgttcggtattttctttaccgacgccgagtccgtgacgtgctatcaggatgtgatggcctgtgacgtggaacgctttaagcgtttcttccatatgatgctggacgaaggtgtttacctggcaccgtcagcgtttgaagcgggctttatgtccgtggcgcacagcatggaagatatcaataacaccatcgatgctgcacgtcgggtgtttgcgaagttgtaaaaggaggaaaaaaaatgaccaagaagcttttagcgctcggtattaaccataaaacggcacctgtatcgctgcgagaacgcgtaacgttttcgccggacacgcttgatcaggcgctggacagcctgcttgcgcagccaatggtgcagggcggggtcgtgctgtcaacctgtaatcgtacagagctgtatctgagcgtggaagagcaggataacctgcaagaagcgctgatccgctggttatgcgattaccataacctgaacgaagacgatctgcgcaacagtctgtactggcatcaggacaatgacgccgtcagccacctgatgcgcgtcgccagcggtctggattcactggtgctgggcgaaccgcaaatcctcggtcaggtgaaaaaagcgtttgcggattcgcaaaaaggccaccttaacgccagcgcgctggagcgaatgttccagaagtctttttccgtcgccaagcgagtgcggactgaaactgatatcggcgccagcgccgtctccgtcgcgtttgccgcctgtacgctcgcccgccaaatctttgaatcgctctcgacggtcactgtactgttagttggcgcgggcgaaactattgaactggtggcgcgtcacctgcgcgagcataaagtacaaaagatgattatcgccaaccgaacccgcgagcgcgcgcaagccctggcggatgaggtaggcgccgaggttatctcgctcagcgatatcgacgcccgtttgcaggatgccgatattatcatcagctcgaccgccagtccgctgccgattatcggcaaaggcatggtggagcgcgcattaaaaagccgtcgtaaccagccgatgctgctggtggatatcgccgtaccgcgcgacgttgagccggaagtcggcaaactggcgaatgcttatctttatagcgtcgatgatttacagagcatcatttcgcataatctggcgcagcgtcaggctgcggcggtagaagcggaaacgattgttgagcaggaagccagcgagtttatggcctggctacgtgcccagggggccagcgagaccattcgggaataccgtagtcagtcggagcagattcgtgacgaactgactaccaaagcgctgtcagcccttcaacagggcggcgatgcgcaagccatcttgcaggatctggcatggaaactgaccaaccgcctgattcatgcgccaacgaaatcacttcaacaggctgcccgtgacggggatgacgaacgcctgaatattctgcgcgacagcctcgggctggagtaactgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaaacccgacgcacaccaggttaaacagtttctgctcaaccttcaggatacgatttgtcagcagctgaccgccgtcgatggcgcagaatttgtcgaagatagttggcagcgcgaagctggcggcggcgggcgtagtcgggtgttgcgtaatggtggtgttttcgaacaggcaggcgtcaacttttcgcatgtccacggtgaggcgatgcctgcttccgccaccgctcatcgcccggaacttgccgggcgcagtttcgaggcgatgggcgtttcactggtagtgcatccgcataacccgtatgttcccaccagccacgcgaatgtgcggttttttattgccgaaaaaccgggtgccgatcccgtctggtggtttggcggtggcttcgacttaaccccattctatggttttgaagaagatgctattcactggcatcgcaccgcccgtgacctgtgcctgccatttggcgaagacgtttatccccgttacaaaaagtggtgcgacgaatacttctacctcaaacatcgcaacgaacagcgcggtattggcgggctgttctttgatgacctgaacacgccagatttcgaccgctgttttgcctttatgcaggcggtaggcaaaggctacaccgacgcttatttaccaattgtcgagcgacggaaagcgatggcctacggcgagcgcgagcgcaatttccagttatatcgtcgcggtcgttatgtcgagttcaatctggtctgggatcgcggcacgctgtttggcctgcaaactggcgggcgcaccgagtctatcctgatgtcaatgccgccactggtacgctgggaatatgattatcagccaaaagatggcagcccagaagcggcgttaagtgagtttattaaggtcagggattgggtgtaa

SEQ ID NO: 5 - DNA, 47 bp, Artificial sequence
  Synthetic DNA
gtgactgggaaaaccctggctaaacccaacttaatcgccttgcagca

SEQ ID NO: 6 - DNA, 49 bp, Artificial sequence
  Synthetic DNA
gtgctttatctgcacggtggcggctaagttatcggctcgatcaacacgc

SEQ ID NO: 7 - DNA, 46 bp, Artificial sequence
  Synthetic DNA
cgatatggaaggagtcggcgattcgtgaagacgaaggcggctgtcg

SEQ ID NO: 8 - DNA, 51 bp, Artificial sequence
  Synthetic DNA
catcacagtcttgctaaagcgcaagcgttggccgattcattaatgcagctg

SEQ ID NO: 9 - DNA, 60 bp, Artificial sequence
  Synthetic DNA
cgacaaaattggagcgttcatccgcgccaacgccgagtaaatcactagtctgaatcggcc

SEQ ID NO: 10 - DNA, 25 bp, Artificial sequence
  Synthetic DNA
catcacagtcttgctaaagcgcaag

SEQ ID NO: 11 - DNA, 20 bp, Artificial sequence
  Synthetic DNA
ggccgattcagactagtgat

SEQ ID NO: 12 - DNA, 59 bp, Artificial sequence
  Synthetic DNA
ttccaccgttgctgttgcgtnnnnnnnnnnnnnnnnntattctgagtcttcgggtgaac

SEQ ID NO: 13 - DNA, 59 bp, Artificial sequence
  Synthetic DNA
cataacgacacaatgctggtnnnnnnnnnnnnnnaagttaaggcggggcaaaaaatagc

SEQ ID NO: 14 - DNA, 59 bp, Artificial sequence
  Synthetic DNA
tagcaccggaaccagttcaannnnnnnnnnnnnnnnaattcgttacaaggggaaatccg

SEQ ID NO: 15 - DNA, 59 bp, Artificial sequence
  Synthetic DNA
gcagcgataaatcggcggaacnnnnnnnnnnnnnnnntgntccgtcaagggatcacggg

SEQ ID NO: 16 - DNA, 46 bp, Artificial sequence
  Synthetic DNA
cactatagggcgaattggagctccatacgcgctgaacgttggtcag

SEQ ID NO: 17 - DNA, 50 bp, Artificial sequence
  Synthetic DNA
tcaagggatcacgggtaggagccaccgatccatgggtaagggagaagaac

SEQ ID NO: 18 - DNA, 22 bp, Artificial sequence
  Synthetic DNA
cactatagggcgaattggagct

SEQ ID NO: 19 - DNA, 20 bp, Artificial sequence
  Synthetic DNA
gttcttctcccttacccatg

SEQ ID NO: 20 - DNA, 21 bp, Artificial sequence
  Synthetic DNA
ccatgggtaagggagaagaac

SEQ ID NO: 21 - DNA, 31 bp, Artificial sequence
  Synthetic DNA
ccggaattcttatttgtatagttcatccatg

SEQ ID NO: 22 - DNA, 59 bp, Artificial sequence
  Synthetic DNA
tctccgaaagtttccatvnnvnnvnnnnngatgsgvnnvnnttttcaccgaaagggttg

SEQ ID NO: 23 - DNA, 59 bp, Artificial sequence
  Synthetic DNA
atcacatcaccgarattgnnnvnnctcvnnvnnvnnctctctccagsttwtttccgcgt

SEQ ID NO: 24 - DNA, 59 bp, Artificial sequence
  Synthetic DNA
gtcggagggacgnnnvnnvnntkgttavnntttrrccycgatgaaaacaacaaatggaa

SEQ ID NO: 25 - DNA, 59 bp, Artificial sequence
  Synthetic DNA
gtcaaaytcrccaamkgatctvnnvnnvnnntgmtgnttnatttaaacgctgaagtcag

SEQ ID NO: 26 - DNA, 59 bp, Artificial sequence
  Synthetic DNA
aaggctatggagatnacvnnrrctgggaavnnvnnvnnvnnccggatcatacgtccgca

SEQ ID NO: 27 - DNA, 59 bp, Artificial sequence
  Synthetic DNA
cataaagtgctggaaaamnatvnnvnnvnnvnnvnnvnnncattantgggccctgacgt

SEQ ID NO: 28 - DNA, 59 bp, Artificial sequence
  Synthetic DNA
acgcttcaccastackdsnttracggcmrwncckcarmtrdgagcacatacctggacgc

SEQ ID NO: 29 - DNA, 59 bp, Artificial sequence
  Synthetic DNA
gcaccaaagatstttcgnvvnvvnntvnnvnnggttttnttwsgcttgacaaactgggt

SEQ ID NO: 30 - DNA, 59 bp, Artificial sequence
  Synthetic DNA
agccgaatccagattattggctgvnnvnnvnnvnnvnntcgttagtagggmvtacggtc

SEQ ID NO: 31 - DNA, 59 bp, Artificial sequence
  Synthetic DNA
gagtgtacgcacamtgcnccaamvnnvnnncavnnvnnvnncagagtcgttwctacaag

SEQ ID NO: 32 - DNA, 70 bp, Artificial sequence
  Synthetic DNA
gaggctgaagcttacgtagaattccaccaccaccaccaccacatgaaagagatcgcggtgacaatagacg

SEQ ID NO: 33 - DNA, 60 bp, Artificial sequence
  Synthetic DNA
tgtagtcagcgatgcaaatgttgaagcgtgcaaaaagtaagcggccgcgaattaattcgc

SEQ ID NO: 34 - DNA, 24 bp, Artificial sequence
  Synthetic DNA
gaggctgaagcttacgtagaattc

SEQ ID NO: 35 - DNA, 20 bp, Artificial sequence
  Synthetic DNA
gcgaattaattcgcggccgc

SEQ ID NO: 36 - DNA, 59 bp, Artificial sequence
  Synthetic DNA
aactttaataaggagggatccataaaaggrrrddddtatatgagtaagtctgaaaatct

SEQ ID NO: 37 - DNA, 58 bp, Artificial sequence
  Synthetic DNA
cgtggtttaagctttggtncancanncvnnvnnvnnntgnaaatggcgcaactggtga

SEQ ID NO: 38 - DNA, 58 bp, Artificial sequence
  Synthetic DNA
cgcctggcccgtggttttnccnntvnnvnnannnntnttaaatttgaagggtgttacc

SEQ ID NO: 39 - DNA, 59 bp, Artificial sequence
  Synthetic DNA
acttataatgatctggctnctvtavnnvnnncanntvngcaatacccgcaagagattgc

SEQ ID NO: 40 - DNA, 58 bp, Artificial sequence
  Synthetic DNA
ggtggtcgtcgtgatgtaatgnatvnnvnnvnnvnnncgngtccggtctatcaggcgg

SEQ ID NO: 41 - DNA, 59 bp, Artificial sequence
  Synthetic DNA
ggtgtttgcgaagttgtaaddrrrrrdddddatgnncvnnvngcttttagcgctcggta

SEQ ID NO: 42 - DNA, 59 bp, Artificial sequence
  Synthetic DNA
aatgacgccgtcagccacntgntgvnnvnnvnnngcngtctggattcactggtgctggg

SEQ ID NO: 43 - DNA, 59 bp, Artificial sequence
  Synthetic DNA
agcgccgtctccgtcgcgnttnccvnnnntvnnvnngcccgccaaatctttgaatcgct

SEQ ID NO: 44 - DNA, 58 bp, Artificial sequence
  Synthetic DNA
attatcgccaaccgaaccngcvagvnnvnnvaanccctggcggatgaggtaggcgccg

SEQ ID NO: 45 - DNA, 58 bp, Artificial sequence
  Synthetic DNA
gcattaaaaagccgtcgtnacvngvnnntgvnnvnngtggatatcgccgtaccgcgcg

SEQ ID NO: 46 - DNA, 53 bp, Artificial sequence
  Synthetic DNA
ccgcataatcgaaattaatannnnnnnntataggggaattgtgagcggataac

SEQ ID NO: 47 - DNA, 54 bp, Artificial sequence
  Synthetic DNA
gtatattagttaagtataagrrrrrrddddddatgaaacccgacgcacaccagg

SEQ ID NO: 48 - DNA, 51 bp, Artificial sequence
  Synthetic DNA
catcgcccggaacttgccnggvnnvnnnncgaggcgatgggcgtttcactg

SEQ ID NO: 49 - DNA, 58 bp, Artificial sequence
  Synthetic DNA
ttctatggttttgaagaagatnctnntnnnnngnatcgcaccgcccgtgacctgtgcc

SEQ ID NO: 50 - DNA, 60 bp, Artificial sequence
  Synthetic DNA
ggcgttaagtgagtttattaaggtcagggattgggtgtaagcacttcgtggccgagctcg

SEQ ID NO: 51 - DNA, 26 bp, Artificial sequence
  Synthetic DNA
tgtttaactttaataaggagggatcc

SEQ ID NO: 52 - DNA, 20 bp, Artificial sequence
  Synthetic DNA
cgagctcggccacgaagtgc

SEQ ID NO: 53 - DNA, 42 bp, Artificial sequence
  Synthetic DNA
gcacttcgtggccgagctcgagtctggtaaagaaaccgctgc

SEQ ID NO: 54 - DNA, 35 bp, Artificial sequence
  Synthetic DNA
ctccttattaaagttaaacaaaattatttctacag

SEQ ID NO: 55 - DNA, 741 bp, Artificial sequence; identical to SEQ ID NO: 2
  Synthetic DNA

SEQ ID NO: 56 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtaaacagttgcgatcaagttaaggcggggcaaaaaatagcgaccatgggtagcaccggaaccagttcaacgtgtacaagcagtggaattcgttacaaggggaaatccgtaaacccgctgcgttatttgccgcagcgataaatcggcggaaccaggcttttgcttgaatgttccgtcaagggatcacgggtaggagccacc

SEQ ID NO: 57 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtctagcccacgcgagaagttaaggcggggcaaaaaatagcgaccatgggtagcaccggaaccagttcaacacgcttgcattttgaaattcgttacaaggggaaatccgtaaacccgctgcgttatttgccgcagcgataaatcggcggaactggtctttctgcttgtatgtccgtcaagggatcacgggtaggagccacc

SEQ ID NO: 58 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtacgttgtggagcgagcgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtcctgattgatctcgaagttaaggcggggcaaaaaatagcgaccatgggtagcaccggaaccagttcaacgccctccgaaaccccaattcgttacaaggggaaatccgtaaacccgctgcgttatttgccgcagcgataaatcggcggaaccaggcttttgcttgaatgttccgtcaagggatcacgggtaggagccacc

SEQ ID NO: 59 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaactctcttgcatttgaaaattcgttacaaggggaaatccgtaaacccgctgcgttatttgccgcagcgataaatcggcggaaccaggcttttgctggattgatccgtcaagggatcacgggtaggagccacc

SEQ ID NO: 60 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaaccgacttgcctctcgcaattcgttacaaggggaaatccgtaaacccgctgcgttatttgccgcagcgataaatcggcggaaccactgtagggcttgagtgttccgtcaagggatcacgggtaggagccacc

SEQ ID NO: 61 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaaccatgtctgttataacaattcgttacaaggggaaatccgtaaacccgctgcgttatttgccgcagcgataaatcggcggaaccttttgcaatcgtatttgttccgtcaagggatcacgggtaggagccacc

SEQ ID NO: 62 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaagagctgtttgcttccgaattcgttacaaggggaaatccgtaaacccgctgcgttatttgccgcagcgataaatcggcggaacacatcagatggggctgtggtccgtcaagggatcacgggtaggagccacc

SEQ ID NO: 63 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaaccagcgtagccattctaattcgttacaaggggaaatccgtaaacccgctgcgttatttgccgcagcgataaatcggcggaaccttgcatgttaataattgatccgtcaagggatcacgggtaggagccacc

SEQ ID NO: 64 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaacacgcttgcattttgaaattcgttacaaggggaaatccgtaaacccgctgcgttatttgccgcagcgataaatcggcggaacgctctatcgtgcggggtggtccgtcaagggatcacgggtaggagccacc

SEQ ID NO: 65 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgttggttatgcactgcggttattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaaagggatagatcaaatgaattcgttacaaggggaaatccgtaaacccgctgcgttatttgccgcagcgataaatcggcggaacagtatgctgctaaacctgttccgtcaagggatcacgggtaggagccacc

SEQ ID NO: 66 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaacacgcttgcattttgaaattcgttacaaggggaaatccgtaaacccgctgcgttatttgccgcagcgataaatcggcggaaccgtgttgttgttggattggtccgtcaagggatcacgggtaggagccacc

SEQ ID NO: 67 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaacacgcttgcattttgaaattcgttacaaggggaaatccgtaaacccgctgcgttatttgccgcagcgataaatcggcggaacggtttttattgttctatgttccgtcaagggatcacgggtaggagccacc

SEQ ID NO: 68 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaacacgcttgcattttgaaattcgttacaaggggaaatccgtaaacccgctgcgttatttgccgcagcgataaatcggcggaaccgcctaccccatttattgttccgtcaagggatcacgggtaggagccacc

SEQ ID NO: 69 - DNA, 741 bp, Artificial sequence
  Synthetic DNA
ccatacgcgctgaacgttggtcagaccttgcaggtgggtaatgcttccggtacgccaatcactggcggaaatgccattacccaggccgacgcagcagagcaaggagttgtgatcaagcctgcacaaaattccaccgttgctgttgcgtcgcaaccgacaattacgtattctgagtcttcgggtgaacagagtgctaacaaaatgttgccgaacaacaagccaactgcgaccacggtcacagcgcctgtaacggtaccaacagcaagcacaaccgagccgactgtcagcagtacatcaaccagtacgcctatctccacctggcgctggccgactgagggcaaagtgatcgaaacctttggcgcttctgaggggggcaacaaggggattgatatcgcaggcagcaaaggacaggcaattatcgcgaccgcagatggccgcgttgtttatgctggtaacgcgctgcgcggctacggtaatctgattatcatcaaacataatgatgattacctgagtgcctacgcccataacgacacaatgctggtccgggaacaacaagaagttaaggcggggcaaaaaatagcgaccatgggtagcaccggaaccagttcaagagtctgtccgtctggaattcgttacaaggggaaatccgtaaacccgctgcgttatttgccgcagcgataaatcggcggaacgagttggggcgacgtttgttccgtcaagggatcacgggtaggagccacc

SEQ ID NO: 70 - protein, 489 aa, Hirudo nipponia
MKEIAVTIDDKNVIASVSESFHGVAFDASLFSPKGLWSFVDITSPKLFKLLEGLSPGYFRVGGTFANWLFFDLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLEKYPTLNKGSLVGPDVGWMGVSYVKGLADGAGDHVTAFTLHQYYFDGNTSDVSTYLDATYFKKLQQLFDKVKDVLKNSPHKDKPLWLGETSSGYNSGTKDVSDRYVSGFLTLDKLGLSAANNVKVVIRQTIYNGYYGLLDKNTLEPNPDYWLMHVHNSLVGNTVFKVDVSDPTNKARVYAQCTKTNSKHTQSRYYKGSLTIFALNVGDEDVTLKIDQYSGKKIYSYILTPEGGQLTSQKVLLNGKELKLVSDQLPELNADESKTSFTLSPKTFGFFVVSDANVEACKK

SEQ ID NO: 71 - protein, 489 aa, Artificial sequence
  Protein translated from synthetic DNA
MKEIAVTIDDKNVIASVSESFHGVAFDASLFSPKGLWSFVDITSPKLFKLLEGLSPGYFRVGGTFANWLFFDLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLEKYPTLNKGSLVGPDVGWMGVSYVKGLADGAGDHVTAFTLHQYYLDGNTSDVSTYLDATYFKKLQQLFDKVKDVLKNSPHKDKPLWLGETSSGYNFGTKDVSDPYLSGFFTLDKLGLSAANNVKVVIRQTIYNGYYGLLDKNTLEPNPDYWLMHVHNSLVGNTVFKVDVSDPTNKARVYAQCTKTNSKHTQSRYYKGSLTIFALNVGDEDVTLKIDQYSGKKIYSYILTPEGGQLTSQKVLLNGKELKLVSDQLPELNADESKTSFTLSPKTFGFFVVSDANVEACKK

SEQ ID NO: 72 - protein, 489 aa, Artificial sequence
  Protein translated from synthetic DNA
MKEIAVTIDDKNVIASVSESFHGVAFDASLFSPKGLWSFVDITSPKLFKLLEGLSPGYFRVGGTFANWLFFDLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLEKYPTLNKGSLVGPDVGWMGVSYVKGLADGAGDHVTAFTLHQYYFDGNTSDVSTYLDATYFKKLQQLFDKVKDVLKNSPHKDKPLWLGETSSGYNSGTKDVSDRYVSGFLTLDKLGLSAANNVKVVIRQTIYNGYYGLLDKNTLEPNPDYWLMHVHNSLVGNTVFKVDVSDPTNKARVYAQCSKTISKRSQSRYYKGSLTIFALNVGDEDVTLKIDQYSGKKIYSYILTPEGGQLTSQKVLLNGKELKLVSDQLPELNADESKTSFTLSPKTFGFFVVSDANVEACKK

SEQ ID NO: 73 - protein, 489 aa, Artificial sequence
  Protein translated from synthetic DNA
MKEIAVTIDDKNVIASVSESFHGVAFDASLFSPKGLWSFVDITSPKLFKLLEGLSPGYFRVGGTFANWLFFDLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLEKYPTLNKGSLVGPDVGWMGVSYVKGLADGAGDHVTAFTLHQYYFDGNTSDVSTYLDATYFKKLQQLFDKVKDVLKNSPHKDKPLWLGETSSGYNSGTKDVSGSRASGFFTLDKLGLSAANNVKVVIRQTIYNGYYGLLDKNTLEPNPDYWLHATPVSLVGTTVFKVDVSDPTNKARVYAQCTKTNSKHTQSRYYKGSLTIFALNVGDEDVTLKIDQYSGKKIYSYILTPEGGQLTSQKVLLNGKELKLVSDQLPELNADESKTSFTLSPKTFGFFVVSDANVEACKK

SEQ ID NO: 74 - protein, 489 aa, Artificial sequence
  Protein translated from synthetic DNA
MKEIAVTIDDKNVIASVSESFHGVAFDASLFSPKGLWSFVDITSPRLDRLARGLSPAFFRVGGTFANWLFFDLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLEKYPTLNKGSLVGPDVGWMGVSYVKGLADGAGDHVTAFTLHQYFFNGSSSAVSTYLDATYFKKLQQLFDKVKDVLKNSPHKDKPLWLGETSSGYNSGTKDVSEGGHAGFLRLDKLGLSAANNVKVVIRQTIYNGYYGLLDKNTLEPNPDYWLMHVHNSLVGNTVFKVDVSDPTNKARVYAQCTKTNSKHTQSRYYKGSLTIFALNVGDEDVTLKIDQYSGKKIYSYILTPEGGQLTSQKVLLNGKELKLVSDQLPELNADESKTSFTLSPKTFGFFVVSDANVEACKK

SEQ ID NO: 75 - protein, 489 aa, Artificial sequence
  Protein translated from synthetic DNA
MKEIAVTIDDKNVIASVSESFHGVAFDASLFSPKGLWSFVDITSPKLFKLLEGLSPGYFRVGGTFANWLFFDLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLEKYIDSLAISLLGPDVGWMGVSYVKGLADGAGDHVTAFTLHQYYFDGNTSDVSTYLDATYFKKLQQLFDKVKDVLKNSPHKDKPLWLGETSSGYNSGTKDVSARAEKGFVTLDKLGLSAANNVKVVIRQTIYNGYYGLLDKNTLEPNPDYWLSDVILSLVGNTVFKVDVSDPTNKARVYAQCAKANADLRQSRYYKGSLTIFALNVGDEDVTLKIDQYSGKKIYSYILTPEGGQLTSQKVLLNGKELKLVSDQLPELNADESKTSFTLSPKTFGFFVVSDANVEACKK

SEQ ID NO: 76 - protein, 489 aa, Artificial sequence
  Protein translated from synthetic DNA
MKEIAVTIDDKNVIASVSESFHGVAFDASLFSPKGLWSFVDITSPKLFKLLEGLSPGYFRVGGTFANWLFFDLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLENNVLLLMVSLMGPDVGWMGVSYVKGLADGAGDHVTAFTLHQYGLDGNPSTKSTYLDATYFKKLQQLFDKVKDVLKNSPHKDKPLWLGETSSGYNSGTKDVSGKVPHGFFRLDKLGLSAANNVKVVIRQTIYNGYYGLLDKNTLEPNPDYWLVPRNKSLVGPTVFKVDVSDPTNKARVYAHCANRVSVVGQSRYYKGSLTIFALNVGDEDVTLKIDQYSGKKIYSYILTPEGGQLTSQKVLLNGKELKLVSDQLPELNADESKTSFTLSPKTFGFFVVSDANVEACKK

SEQ ID NO: 77 - protein, 489 aa, Artificial sequence
  Protein translated from synthetic DNA
MKEIAVTIDDKNVIASVSESFHGVAFDASLFSPKGLWSFVDITSPKLFKLLEGLSPGYFRVGGTFANWLFFDLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLEKYPTLNKGSLVGPDVGWMGVSYVKGLADGAGDHVTAFTLHQYYFDGNTSDVSTYLDATYFKKLQQLFDKVKDVLKNSPHKDKPLWLGETSSGYNSGTKDVSWCSRGGFFSLDKLGLSAANNVKVVIRQTIYNGYYGLLDKNTLEPNPDYWLARVVSSLVGSTVFKVDVSDPTNKARVYAHCSKRTSGTAQSRYYKGSLTIFALNVGDEDVTLKIDQYSGKKIYSYILTPEGGQLTSQKVLLNGKELKLVSDQLPELNADESKTSFTLSPKTFGFFVVSDANVEACKK

SEQ ID NO: 78 - protein, 489 aa, Artificial sequence
  Protein translated from synthetic DNA
MKEIAVTIDDKNVIASVSESFHGVAFDASLFSPKGLWSFVDITSPKLFKLLEGLSPGYFRVGGTFANWLFFDLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLEKYPTLNKGSLVGPDVGWMGVSYVKGLADGAGDHVTAFTLHQYYFDGNTSDVSTYLDATYFKKLQQLFDKVKDVLKNSPHKDKPLWLGETSSGYNSGTKDVSDRYVSGFLTLDKLGLSAANNVKVVIRQTIYNGYYGLLDKNTLEPNPDYWLMHVHNSLVGNTVFKVDVSDPTNKARVYAHCSNPMALLMQSRYYKGSLTIFALNVGDEDVTLKIDQYSGKKIYSYILTPEGGQLTSQKVLLNGKELKLVSDQLPELNADESKTSFTLSPKTFGFFVVSDANVEACKK

SEQ ID NO: 79 - protein, 489 aa, Artificial sequence
  Protein translated from synthetic DNA
MKEIAVTIDDKNVIASVSESFHVTIGDAGNFSPKGLWSFVDITSPKLVLLLTLLSPGYFRVGGTGIILLAFGLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLEKYPTLNKGSLVGPDVGWMGVSYVKGLADGAGDHVTAFTLHQYYFDGNTSDVSTYLDATYFKKLQQLFDKVKDVLKNSPHKDKPLWLGETSSGYNSGTKDLSTSTTAGFLRLDKLGLSAANNVKVVIRQTIYNGYYGLLDKNTLEPNPDYWLMHVHNSLVGNTVFKVDVSDPTNKARVYAHCTNRGPAAAQSRYYKGSLTIFALNVGDEDVTLKIDQYSGKKIYSYILTPEGGQLTSQKVLLNGKELKLVSDQLPELNADESKTSFTLSPKTFGFFVVSDANVEACKK

SEQ ID NO: 80 - protein, 489 aa, Artificial sequence
  Protein translated from synthetic DNA
MKEIAVTIDDKNVIASVSESFHGVAFDASLFSPKGLWSFVDITSPKLGVLAPSLSPGYFRVGGTIIRLLIFNLDENNKWKDYWAFKDKTPETATITRRWLFRKQNNLKKETFDDLVKLTKGSKMRLLFDLNAEVRTGYEIGKKMTSTWDSSEAEKLFKYCVSKGYGDNIDWELGNEPDHTSAHNLTEKQVGEDFKALHKVLEKYPTLNKGSLVGPDVGWMGVSYVKGLADGAGDHVTAFTLHQYYFDGNTSDVSTYLDATYFKKLQQLFDKVKDVLKNSPHKDKPLWLGETSSGYNSGTKDVSDRYVSGFLTLDKLGLSAANNVKVVIRQTIYNGYYGLLDKNTLEPNPDYWLGVQEASLVGSTVFKVDVSDPTNKVRVYAHCPKDLPLGIQSRFYKGSLTIFALNVGDEDVTLKIDQYSGKKIYSYILTPEGGQLTSQKVLLNGKELKLVSDQLPELNADESKTSFTLSPKTFGFFVVSDANVEACKK

(19) United States
(12) Patent Application Publication
(10) Pub. No.: US 2016/0319272 A1
(43) Pub. Date: Nov. 3, 2016

(54) Method for Directed DNA Evolution using Combinatorial DNA Libraries

(71) Applicants: Zhen Kang, Wuxi (CN); Jian Chen, Wuxi (CN); Guocheng Du, Wuxi (CN); Peng Jin, Wuxi (CN)

(72) Inventors: Zhen Kang, Wuxi (CN); Jian Chen, Wuxi (CN); Guocheng Du, Wuxi (CN); Peng Jin, Wuxi (CN)

(73) Assignee: Jiangnan University, Wuxi (CN)

(21) Appl. No.: 15/140,408

(22) Filed: Apr. 27, 2016

(30) Foreign Application Priority Data
Apr. 29, 2015 (CN) 201510212925.7

(51) Int. Cl. C12N 15/10

(52) U.S. Cl. CPC C12N 15/1027; C12N 15/1058

(57) ABSTRACT

The present invention provides a method of rapid directed DNA evolution based on single-stranded combinatorial DNA mutant library. The single- and double-stranded mutant library are constructed either separately or simultaneously using editing primers that contain mutated nucleotides and are targeted to different regions of the parent DNA sequence. The mutant library is then inserted into expression vectors and mutants with desired property are obtained by high throughput screening. Evolution of promoters, enzymes and metabolic pathways were successfully achieved using this method and mutants with excellent properties were obtained. The method of the present invention is simple, rapid, and efficient. It can be used for directed evolution of regulatory sequence such as promoters and ribosome binding sites, and is especially suitable for introducing diverse mutations into protein encoding genes, leading to rapid directed evolution of gene of interest.

### CROSS-REFERENCES AND RELATED APPLICATIONS

This application claims the benefit of priority to Chinese Application No. 201510212925.7, entitled “A Method for Directed DNA Evolution using Combinatorial DNA Libraries”, filed Apr. 29, 2015, which is herein incorporated by reference in its entirety.

### BACKGROUND OF THE INVENTION

1. Field of the Invention

The present invention relates to the field of genetic engineering, and more particularly relates to a method of rapid and directed DNA evolution using combinatorial DNA libraries.

2. Description of the Related Art

With the development of molecular biology and genetic engineering, thousands of natural enzymes have been found. Because of many benefits of using enzymes, such as environmental friendliness, high specificity and mild reaction conditions, they have been widely used in the synthesis of chemical intermediates, complex drugs, and even bio fuels. Enzymes are the core participants of synthetic biology and metabolic engineering. Complex compounds which cannot be synthesized by organic synthesis methods, such as aromatic hydrocarbons, carbohydrates, organic acids, alcohols and other secondary metabolites, can be synthesized through the construction of a series of microbial metabolic synthesis pathway. However, in most cases, native enzymes usually have certain limitations in their properties, which greatly limits their use in a variety of complex conditions.

Highly precise and efficient enzymes have been evolved in the processes of dynamic equilibrium between genetic mutation and natural selection. As natural selection is a very slow and time consuming process, there is an urgent need for developing methods for artificial evolution and transformation of native enzymes to obtain novel enzymes with desired properties. Currently, physical and chemical methods have been developed to improve the frequency of gene mutation. For example, ultraviolet rays and nitrite have been used to greatly increase the mutation frequency, making it possible for artificial and rapid evolution of genes. However, mutations caused by physical and chemical methods are often deleterious and random ones, which can not meet the needs of large-scale evolution. With advancements in molecular biology techniques, introduction of specific or random mutations to native genes at the level of genetic manipulation has become possible. The random mutations are achieved by introducing random base substitutions into a gene and screening for desired mutants. For example, error-prone PCR, a widely used technology for gene evolution, is performed by randomly introduced point mutations during polymerase chain reactions (PCR) and genes with desirable mutations are screened by high-throughput screening techniques. After several rounds of operations, novel enzymes with significantly different properties and functions can be obtained. A series of PCR-based methods for gene evolution have been developed, such as site-directed mutagenesis, chimeric gene mutation, and random chimeragenesis at transient templates. However, the mutation sites generated by these methods are not very diverse and the mutants contain mostly deleterious mutations, and the methods are laborious and with low efficiency.

In 1994, Stemmer proposed a new technologies for directed evolution and published an article titled “Rapid evolution of a protein in vitro by DNA shuffling” in the scientific journal, Nature. Using DNA shuffling, a new strain was obtained after three rounds of screening and two cycles of backcrossing, whose minimum inhibitory concentration of cefotaxime was 16,000 times higher than that of the original strain, much higher than that of the mutations found in the error-prone PCR mutation screening. Based on DNA shuffling techniques, more methods have been developed to improve the hybridization and gene screening efficiency, which have achieved good results. However, DNA shuffling techniques require high sequence homology (greater than 75%) and homologous genetic materials, which limits the widespread application of DNA shuffling technology, and mutants produced by DNA shuffling techniques are mostly frameshift mutants.

Currently, transformation of biosynthetic pathways mostly focuses on the regulation the expression of key genes in biosynthetic pathways, such as enhancing the expression of pathway genes, knocking out or inhibiting competitive pathways, optimizing the promoter or ribosome binding sites for genes of interest, using codon-optimized genes, and altering the interval regulatory regions of pathway genes. Merely increasing the expression level of selected enzymes may lead to a waste of energy in cell synthesis and cast a big burden to cell growth and metabolism, and can not fundamentally solve the problem of metabolic pathways flow balance. Therefore, improvement on the properties of pathway enzymes is very neccessary, for example, to solve the rate-limiting problems of key enzymes due to product or substrate inhibition.

The present invention provides a method for rapid and efficient evolution of a target DNA, which comprises synthesizing a single-stranded DNA library, which is used to obtained a dsDNA mutant library with great diversity using PCR techniques, and screening for mutant DNA with desired property from the DNA mutant library. The method of the present invention overcomes the problem of lack of diversity in the random mutation methods, and the limitation of requiring homologous sequences in the DNA shuffling method. The method is easy to operate and can generate DNA mutant libraries with great diversity, which lays the foundation for rapid in vitro gene evolution. Combined with protein structural bioinformatics and synthetic biology, it is particularly suitable for applying to directed and rapid evolution of enzymes and synthetic metabolic pathways.

### DETAILED DESCRIPTION

The present invention provides a method for rapid and efficient combinatorial DNA evolution based on single-stranded DNA mutant libraries, defined as Rapid and Efficient Combinatorial Oligonucleotides for Directed Evolution (RECODE). The method comprises preparation of mutant libraries and screening of mutants with desired properties from the mutant libraries, wherein the mutant libraries include single- and double-stranded DNA mutant library. The single- and double-stranded mutant library can be constructed separately or simultaneously. For construction of the single-stranded mutant library, multiple oligonucleotides (primers) are designed to anneal to different target regions of a parent DNA strand, wherein each oligonucleotide has one or more mutations comparing to the corresponding parent sequence. The mutagenic single-stranded oligonucleotides are annealed to the parent template DNA, which are extended from 5′ to 3′ by a DNA polymerase until it reaches an adjacent oligonucleotide. The adjacent 5′ phosphate and 3′ hydroxyl termini were ligated by a Taq DNA ligase to form a duplex DNA structure, which contains a parent DNA strand and a mutant DNA strand. The parent DNA template strand is thus used to generate a library of single-stranded mutant DNAs with combinatorial mutations introduced by different mutagenic oligonucleotides. For the construction of double-stranded mutant library, additional anchor sequences are introduced to the both ends of single-stranded mutant DNAs, which can be used to design PCR primers for specifically converting the single-stranded mutant DNAs into double-stranded mutant DNA library.

The target region of the parent gene for mutation can be specifically chosen or randomly generated. The number of target regions for mutation is not limited, and it can be set based on actual needs. For example, the number of target regions can be 1˜15 in a gene with less than 2000 bp.

The direction of the primers with mutations can be either-3′→5′ or 5′→3′ direction as compared to the parent gene, but not both. When the parent gene is a double-stranded DNA, the primers with the same direction ensures that only one strand with the specific direction is synthesized and a full-length single-stranded DNA with combinatorial integration of different mutations can be obtained via DNA polymerization and ligation.

The parent gene can be DNA regulatory sequences such a promoter, a ribosome binding site, or other regulatory element for regulating gene expression, or it can a gene encoding a protein such as an enzyme or a key protein of a metabolic pathway. The form of the parent gene is without limitation, which can be plasmids, genomic sequences, double-stranded DNAs or single-stranded DNAs.

For construction of single-stranded mutant library, primers, which are termed as editing primers, are designed to anneal to one or more targeted mutation regions of the parent gene. The editing primers contain mutated nucleotide(s) that are to be introduced into the target region, together with appropriate length of nucleotide sequence that are complementary to the target region of the parent gene. When there are multiple target regions, all the editing primers have the same direction, that is, they all bind to the same strand of the parent gene and extend to add nucleotide in the same direction. Additionally, upstream and downstream anchor primers with the same direction as the editing primers are also designed, wherein the upstream anchor primer anneals to an region located upstream (5′ terminal) of all the editing primers, and it contains a 15˜25 nt anchor sequence at its 5′ end and wherein the downstream anchor primer anneals to an region located downstream (3′ terminal) of all the editing primers, and it contains a 15˜25 nt anchor sequence at its 3′ end. The mutations introduced in the editing primers can be specific nucleotides or degenerate ones.

In one embodiment of the present invention, the single- and double-stranded mutant library are constructed in the same PCR system. PCR end primer pairs, which specifically anneal to the 3′ and 5′ end anchor sequences of the single-stranded DNA mutant, are added along with the editing primers and anchor primers in the PCR system for the construction of the single-stranded mutant library. In this way, once a full-length mutant strand with anchor sequences is generated, it can be used as a template to generate a double-stranded DNA mutant.

In one embodiment of the present invention, the single-stranded mutant library and double-stranded mutant library are constructed separately. The construction of double-stranded mutant library can be carried out in two ways. In one embodiment, PCR end primer pairs are directly added to the PCR system that has been used to construct the single-stranded mutant library without further purification. The double-stranded mutant DNA library is generated using the single-stranded mutant DNAs as templates. In another embodiment, column-purified single-stranded mutant DNAs are used as the template for generating the double-stranded mutant DNA library.

In one preferred embodiment, the editing primer is generally designed to be less than 59 nt, which can be increased if needed. A long editing primer can be divided into two short primers. The mutated nucleotides are typically placed in the middle portion of the editing primer, and can be highly degenerate if needed. In order to avoid termination codons TAA, TAG and TGA, triplet codons can be degenerated to VNN so that the probability of having a terminator in a protein coding region can be reduced. The editing primer is preferably purified by PAGE purification method.

In one embodiment of the present invention, the anchor primer can serve the function of editing primer when it contains mutated nucleotides to be introduced into the target region.

The anchor sequence on the anchor primers serves two functions. Firstly, it is used as the single-stranded mutant DNA-specific sequence that can be used to design PCR primers for construction of double-stranded mutant libraries. Secondly, it is used as homologous sequence for inserting the double-stranded DNA mutants into expression vectors via homologous recombination. Because using highly degenerate nucleotides in the mutation may introduce additional restriction sites internal to the gene, it is not recommended to use restriction enzymes for inserting the double-stranded mutants into the expression vectors.

In one embodiment of the present invention, the hydroxyl groups at the 5′ end of editing primers and downstream anchor primer are phosphorylated by T4 polynucleotide kinase.

In one embodiment of the present invention, editing primers have 15˜30 nt of oligonucleotides at the 3 and 5′ ends that are 100% complementary to the sequences in the parent DNA.

In one embodiment of the present invention, for constructing the single-stranded mutant library, DNA extension and DNA ligation are performed in the same reaction mixture. Upstream and downstream anchor primers, editing primers, parent DNA template are first mixed in an appropriate molar ratio, and DNA polymerase, DNA ligase and reaction buffer are added to the DNA mixture. The reaction buffer contains 1˜10 mM Mg²⁺; the annealing temperature for PCR is 40˜66° C., the elongation temperature for PCR is 65˜72° C. and the reaction cycle for PCR is 1˜35. The final products in the PCR system contain a large amount of single-stranded DNAs with mutation, mutated DNA in a double-stranded state that are bound with the parent DNA template, and a single-stranded parent DNA that is not used as a template. Only the DNA mutants have unique anchor sequences at the 3′ and 5′ ends.

In one embodiment of the present invention, equal molar editing primers that bind to different target regions are used to construct the single-stranded mutant library.

In one embodiment of the present invention, the single-stranded mutant library is constructed in the first round PCR. The unpurified PCR products containing the single-stranded mutant library or column-purified single-stranded mutant library is then used as a template in the second round of PCR to generate a double-stranded mutant library. An end primer pair is designed to anneal to the anchor sequences that are added to the ends of the single-stranded mutant DNA during the first round PCR. The end primer pair is used to specifically amplify the single-stranded mutant DNA with anchor sequences and convert the single-stranded mutant DNA library into the double-stranded DNA mutant library.

In another embodiment, the single- and double-stranded mutant DNA libraries are generated in the same PCR system (hereafter referred as one-step PCR), wherein the end primer pair are added to the PCR system used to construct the single-stranded mutant library as described above. Once the single-stranded mutant DNA with anchor sequences is generated, it can be used as the template for generation of the double-stranded mutant DNA using the end primer pair as the primer. The double-stranded mutant library is purified and inserted into expression vectors to build a high throughput expression library, which can then be used to screen for mutants with desirable properties.

In one embodiment of the present invention, after the preparation of single-stranded mutant library, DpnI endonuclease can be used to digest the parent DNA before the preparation of double-stranded mutant library. Even without digestion of parent DNA with DpnI endonuclease, amplification of parent DNA will not occur because the end primer pair only anneal to the anchor sequences on the single-stranded mutant DNA but not to the parent DNA.

In one embodiment of the present invention, when the PCR mixture containing the single-stranded mutant library or column-purified single-stranded mutant library is used as templates in the second round of PCR, a large amount of double-stranded DNA mutants can be generated after a few PCR cycles.

In one embodiment of the present invention, the single- and double-stranded mutant library is constructed by one-step PCR, comprising the following steps: upstream and downstream anchor primers, editing primers, and parent DNAs are first mixed in an appropriate molar ratio; the end primer pair (2-5-fold of the amount of upstream and downstream anchor primers) are then added to the mixture; and DNA polymerase, DNA ligase and reaction buffer are added at last, wherein the reaction buffer contains 1˜10 mM Mg^{2±}, the annealing temperature for PCR is 40˜66° C., the elongation temperature for PCR is 65˜72° C. and the reaction cycle for PCR is 1˜35. The final reaction mixture contains a large amount of double-stranded mutants and a very small amount of parent DNA.

In the one-step PCR, to minimize the binding of upstream/downstream anchor primers to the end primer pair during the annealing process, the length of the anchor sequence is about one-third of the length of the anchor primers.

In one embodiment, the upstream (or downstream) anchor primer has 60 nucleotides, 40 nt of which are complementary to the parent DNA template and the remaining 20 nt is the anchor sequence, and the end primer has 20 nucleotides that completely match to the 20 nt anchor sequence.

In one embodiment of the present invention, the ratio of the added amount of upstream (or downstream) anchor primer to that of the end primer is 1: (2˜5). The excess end primers ensure that the generated single-stranded mutants are completely converted into double-stranded mutants.

In one embodiment of the present invention, the DNA polymerase used in the PCR is a thermostable, high-fidelity DNA polymerase, preferably a high-fidelity DNA polymerase without 5′-3′ exonuclease activity, such as Phusion DNA polymerase (New England Biolabs, Ipswich, Mass.).

In one embodiment of the present invention, the DNA ligase is a thermostable DNA ligase, such as Taq DNA ligase (New England Biolabs), 9N DNA ligase (New England Biolabs) and Ampligase (Illumina, San Diego, Calif.), preferably Ampligase.

In one embodiment of the present invention, the mutant library is inserted into expression vectors by in vitro recombinant DNA techniques. The recombinant DNA techniques includes, but not limited to, fusion PCR, Gibson assembly, T4 DNA polymerase-mediated recombinant techniques and in vivo yeast assembly techniques. Gibson assembly is used to assemble dsDNA fragments with homologous end sequences. The principle of Gibson assembly is as follows: T5 exonuclease cuts from the 5′ end of double-stranded DNA fragments, resulting in a 3′ sticky end. With homologous sequences contained in each DNA fragment, the complementary sticky ends of two DNA fragments bind to each other, the gaps are filled by DNA polymerases and the nicks are ligated by DNA ligases, achieving a seamless assembly of multiple DNA fragments.

FIG. 1 shows an exemplary embodiment to illustrate the principles of the present invention. This method includes two rounds of PCR and a screening process. The first round of PCR involves five primers all having the same direction (5′ to 3′) as shown in FIG. 1. The two lines containing dotted lines represent the anchor primers, and the dotted lines represent the anchor sequences. The lines with ★, ▴ and ⋄ represent editing primers designed for different target regions. In the denaturing step of the PCR process, the parent DNA was separated into two single-stranded DNAs. Since all the five primers can only bind 3′ to 5′ direction strand, only the strands with a 3′ to 5′ direction can be used as templates. As a result, the final PCR mixture contains a large amount of single-stranded DNA mutants with a 5′ to 3′ direction, a small amount of single-stranded parent DNA which are not used as templates and DNA mutants in double-stranded state which are bound to the parent DNA templates. The second round of PCR was preformed using PCR product mixture obtained from the first PCR or single-stranded mutants purified by DNA columns as the templates. The reaction system for the second PCR can be unchanged from the first PCR and the end primer pairs is directly added into the reaction system of the first PCR, or a new PCR system can be set up using the end primer pairs as the primer and single-stranded mutants from the first PCR as the template. The forward and backward end primers are designed to match the anchor sequences on both ends of the single-stranded mutant DNAs, which ensures that only the single-stranded DNA with anchor sequence can be amplified during the second PCR while the parent DNA cannot. Finally, desired mutants can be obtained by screening the expression library containing double-stranded mutants.

FIG. 2 shows the principle of one-step PCR for construction of the single- and double-stranded mutant library in one reaction system, which is a simpler procedure developed upon the method shown in FIG. 1. In this method, the end primer pair is directly added to the PCR system of the first PCR in FIG. 1. During the PCR process, the newly generated single-stranded mutants can be used as templates to generate double-stranded mutants using the end primers, while the parent strand cannot be amplified due to lack of anchor sequences. As a result, mutant DNAs were produced at an exponential growth rate, a much faster rate than that of the two-step PCR. The one-step PCR is more convenient to operate and saves more time.

The present invention provides a rapid and efficient method for directed DNA evolution based on single-stranded combinatorial DNA mutant library. The method uses PCR techniques and editing primers with mutated nucleotide(s) to synthesize single-stranded mutant DNAs with different combinations of mutated sites. The single-stranded mutant DNAs are converted to a double-stranded mutant DNA library which are inserted into expression vectors for screening of desirable mutants.

The method of the present invention is a simple (one or two rounds of PCR with any form of DNA templates), rapid (one-round evolution of 5 kb DNA within 3 hours), efficient, and powerful in vitro evolution method. It overcomes the problem of lack of diversity in the random mutation methods and the limitation of requiring homologous sequences in the DNA shuffling methods. It is simple to operate and can generate mutant libraries with great diversity, which is suitable for rapid directed evolution of DNA sequence in vitro. The parent DNA used for the directed evolution can be of any kind, including promoters, ribosome binding sites, the non-transcriptional regulatory regions, and protein encoding genes. The present invention is of course applicable for making only one targeted mutation.

### BRIEF DESCRIPTION OF DRAWINGS

FIG. 1. Schematic diagram of RECODE with two-step PCR. Dotted lines represent the anchor sequences.

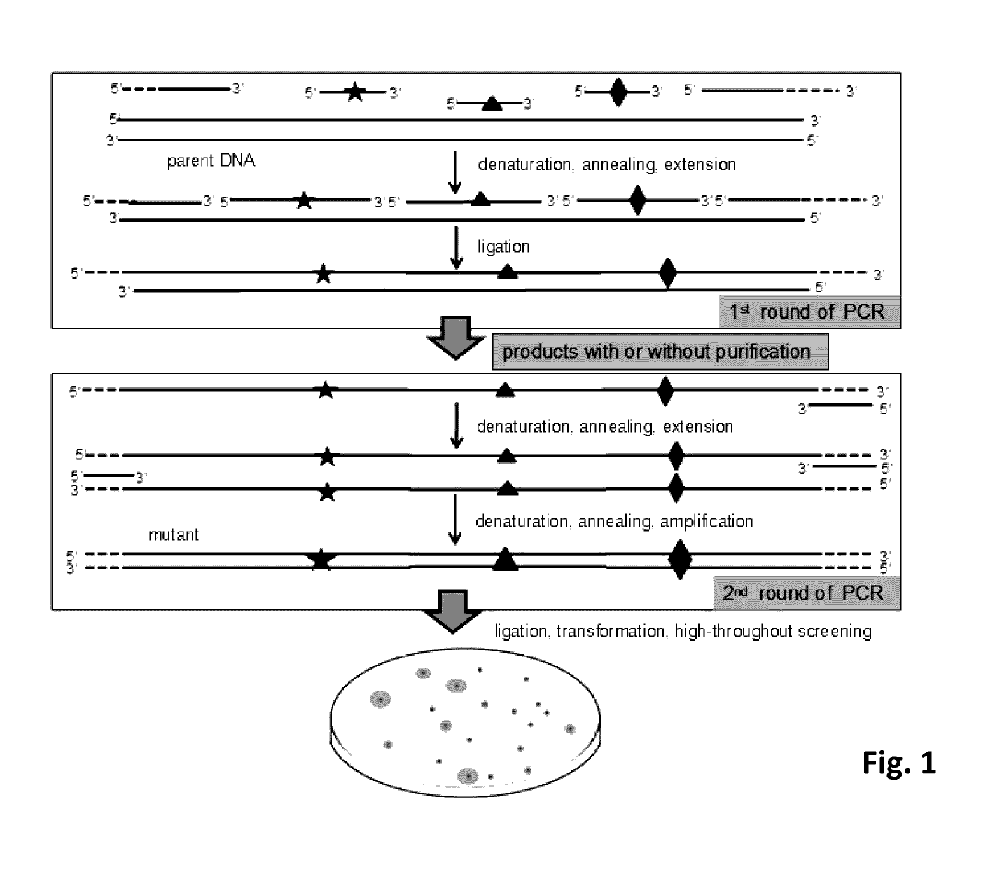

FIG. 2. Schematic diagram of RECODE with one-step PCR. Dotted lines represent the anchor sequences.

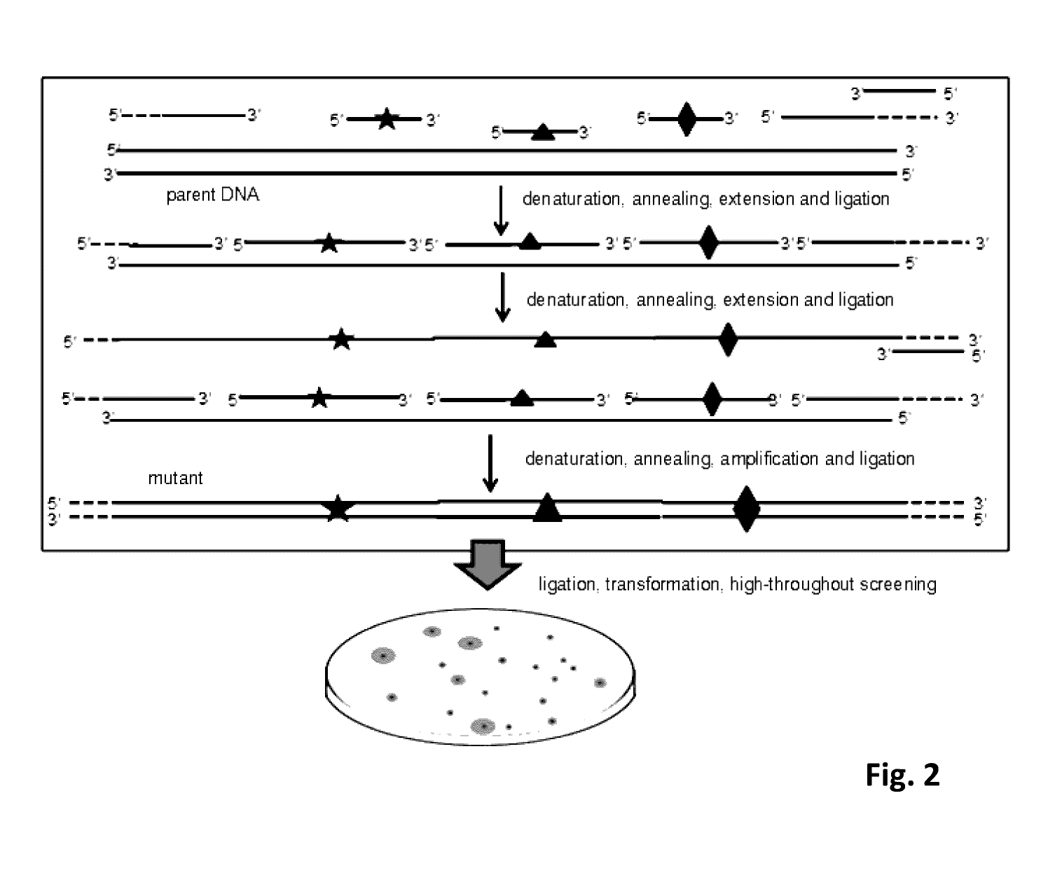

FIG. 3. The phenotype of the recombinants with co-expression of wild-type β-galactosidase and esterase.

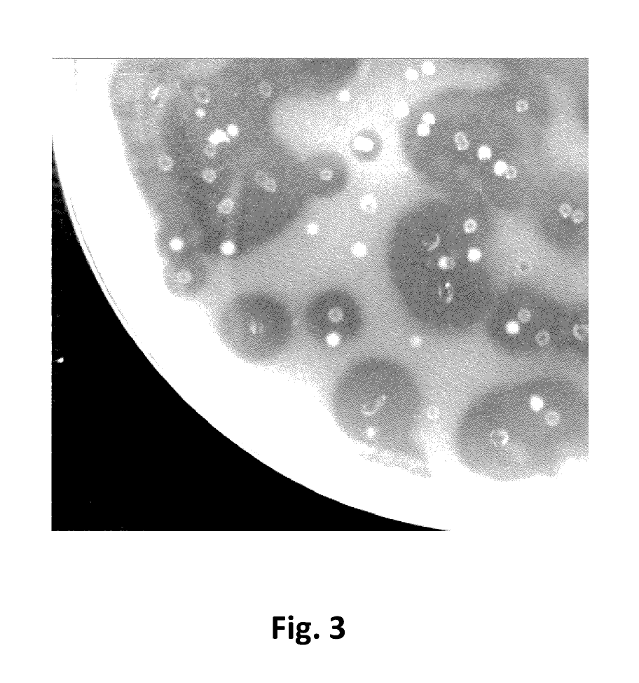

FIG. 4. The phenotype of the recombinants with combinatorial mutation of LacZ-Esterase after rapid evolution by RECODE using two-step PCR systems. The purified single-stranded mutant library is used as templates in the second PCR.

FIG. 5. The phenotype of the recombinants with combinatorial mutation of LacZ-Esterase after rapid evolution by RECODE using two-step PCR systems. The final products of the first PCR without purification is directly used as templates in the second PCR.

FIG. 6. The phenotype of the recombinants with combinatorial mutation of LacZ-Esterase after rapid evolution by RECODE with one-step PCR systems.

FIG. 7. The specific fluorescence units of the rpoS mutants; arrows indicated the specific fluorescence units of the wild type promoter rpoS.

FIG. 8. Alignment of the nucleic acid sequences of the rpoS mutants with different transcriptional strength. The sequences included in the alignment are wild type rpoS (SEQ ID NO: 55) and mutants A5 rpoS (SEQ ID NO: 56), D5 rpoS (SEQ ID NO: 57), B1 rpoS (SEQ ID NO: 58), D3(SEQ ID NO: 59), G4(SEQ ID NO: 60), B7(SEQ ID NO: 61), E8(SEQ ID NO: 62), B12(SEQ ID NO: 63), E8a (SEQ ID NO: 64), A6(SEQ ID NO: 65), H10(SEQ ID NO: 66), F7(SEQ ID NO: 67), F11(SEQ ID NO: 68) and A2(SEQ ID NO: 69). The numbers below the sequence alignment indicate the position of the particular nucleotide in the original sequences. The shaded sequences indicate the mutant region.

FIG. 9. Relative enzyme activities of the HAase and its mutants.

FIG. 10. Diverse distribution of the mutant amino acids in the constructed mutants with altered enzyme activities. The amino acid sequences included in the alignment are wild type LHyal(SEQ ID NO: 70) and mutants M2D7(SEQ ID NO: 71), M4A4(SEQ ID NO: 72), M5B4(SEQ ID NO: 73), M2G1(SEQ ID NO: 74), M2E7(SEQ ID NO: 75), M2F7(SEQ ID NO: 76), M2G7(SEQ ID NO: 77), M4A12(SEQ ID NO: 78), M2H3(SEQ ID NO: 79), and M1F6(SEQ ID NO: 80). The shaded sequences indicate mutant residues.

FIG. 11. Construction of the hemL-hemA-hemF gene of ALA synthesis pathway in E. coli.

FIG. 12. ALA accumulation of the mutants with mutated pathway elements; LAF was the control recombinant plasmid and A1-A5 were mutants with different ALA accumulation.

### EXAMPLES

### Information of Related Nucleotide Sequences

SEQ ID NO:1 is the nucleotide sequence of fusion fragment of LacZ promoter, β-galactosidase gene lacZ, Esterase.

SEQ ID NO:2 is the nucleotide sequence of the constitutive promoter rpoS from E. coli.

SEQ ID NO:3 is the nucleotide sequence of hyaluronidase gene LHyal.

SEQ ID NO:4 is the nucleotide sequence of the reconstructed ALA synthesis pathway gene hemL-hemA-hemF.

### Example 1

### Construction of Combinatorial Mutant Library by RECODE with Two-Step PCR Systems

(A) Preparation of Double-Stranded Mutant Library with Purified Single-Stranded Mutant Library

Combinatorial lethal mutation of lacZ-Esterase (containing a β-galactosidase gene lacZ and an Esterase) with a nucleotide sequence of SEQ ID NO:1 was used in this example. The diversity of the mutant library can be indicated by the phenotype on screening plates with 0.1 mM isopropyl-β-D-thiogalactopyranoside (IPTG), 20 μg/ml X-Gal and 1% tributyrin. The lacZ and Esterase gene were recombined into plasmid pMD19 and transformed into E. coli JM109, resulting in a recombinant strain with a blue colony (when X-gal is depredated by lacZ) and the transparent zone (when tributyrin is hydrolyzed by Esterase) on the screening plate.

The Esterase gene was connected to the downstream of the β-galactosidase gene lacZ (with a LacZ promoter). Three editing primers (JPS-MD/LacZ-P1F, JPS-MD/Est-P2F and JPS-MD/Est-P3F) were designed for combinatorial mutation, whose target regions were one site of the internal lacZ and two sites of the internal Esterase, respectively. A pair of anchor primers (JPS-LZE/UTA and JPS-LZE/DTA) and a pair of end primers (LZE-F and LZE-R) were designed. The nucleotide sequences of primers were as follows:

JPS-MD/LacZ-P1F:
(SEQ ID No: 5)
GTGACTGGGAAAACCCTGGCTAAACCCAACTTAATCGCCTTGCAGCA
JPS-MD/Est-P2F:
(SEQ ID No: 6)
GTGCTTTATCTGCACGGTGGCGGCTAAGTTATCGGCTCGATCAACAC
GC
JPS-MD/Est-P3F:
(SEQ ID No: 7)
CGATATGGAAGGAGTCGGCGATTCGTGAAGACGAAGGCGGCTGTCG
JPS-LZE/UTA:
(SEQ ID No: 8)
CATCACAGTCTTGCTAAAGCGCAAGCGTTGGCCGATTCATTAATGCA
GCTG
JPS-LZE/DTA:
(SEQ ID No: 9)
CGACAAAATTGGAGCGTTCATCCGCGCCAACGCCGAGTAAATCACTA
GTCTGAATCGGCC
LZE-F:
(SEQ ID No: 10)
CATCACAGTCTTGCTAAAGCGCAAG
LZE-R:
(SEQ ID No: 11)
GGCCGATTCAGACTAGTGAT

Mutations (underlined) were introduced to the middle of the three editing primers by replacing the original triplet codons from GTT to TAA, TAC to TAA and ATGA to -TGA, respectively. Introduction of any of the terminators will lead to premature termination of translation, resulting in deactivation of the corresponding enzymes. The anchor primers (JPS-LZE/UTA and JPS-LZE/DTA) have the same direction as the editing primers, and they can anneal to the same template strand. In addition, there was an anchor sequence (more than 20nt) in the anchor primer, which has the same sequence as the gap sequence of the cloning vector and can be used for homologous recombination cloning (TA cloning technology was used in the this example).

The three editing primers and the downstream anchor primer JPS-LZE/DTA were mixed at the same concentration and phosphorylated by T4 polynucleotide kinase according manufacturer's instruction. The reaction was performed at 37° C. for 30 min and the polynucleotide kinase was subsequently heat inactivated at 70° C. for 10 min.

Preparation of single-stranded DNA library was carried out by synchronous PCR cycling reaction of DNA extension and ligation. The 50 μl system contained 5 μl 10× reaction buffer, the upstream anchor primer JPS-LZE/UTA, the phosphorylated downstream anchor primer, appropriate amount of the phosphorylated editing primers (calculated according to the phosphorylation system, 5 pmol of each editing primer was added), 1˜5 ng DNA template (the ratio of template to primer was 1:100), 0.5 μl Phusion DNA polymerase (NEB) and 1 μl Ampligase thermostable DNA ligase (EPI). The system was performed with the following PCR conditions: 2 min at 94° C.; 25 cycles of 30 s at 94° C., 1 min at 50° C. and 5 min at 66° C.; 5 min at 72° C.; a hold period at 4° C. The products of the first round of PCR was single-stranded DNA library, and the products can be used for further experiments immediately or stored at −20° C.

Preparation of double-stranded DNA library was carried out by applying the products of the first PCR as the templates in the second round of PCR and complete full-length gene can be obtained. The 50 μl reaction system contained 25 μl 2× premixed DNA polymerase (Superpfu mix, purchased from Hangzhou-Biotech Co., Ltd), end primers (LZE-F and LZE-R each 1 μl, 10 μM) for amplifying full-length gene and column purified single-stranded templates from the first PCR. The reaction system was performed with the following PCR conditions: 3 min at 94° C.; 3 cycles of 30 s at 94° C., 30 min at 55° C. and 1 min at 68° C.; 5 min at 72° C.; a hold period at 4° C. After that, 0.5 μl Taq DNA polymerase was added to the mixture and hold at 72° C. for 10 min to add an extra Adenine nucleotide to the 3′ end of the products for the subsequent TA-cloning. PCR products were run on 1% agarose gels, bands of the correct size were excised and purified with a gel extraction kit, according to the manufacturer's protocol.

T vector kit pMD19-T (Takara Bio) was used for the cloning of the mutant library. 50 ng pMD19-T was added with equal molar of mutant fragments. The reaction system was mixed and held at 16° C. overnight for ligation and the ligated products were transformed to competent E. coli cells with normal transformation steps. After cultivated at 16° C. for 1 hour, all of the transformed cells were cultured on LB plates supplemented with 100 μg·ml⁻¹ ampicillin, 0.1 mM IPTG, 20 μg·ml⁻¹ X-gal and 1% tributyrin overnight at 37° C.

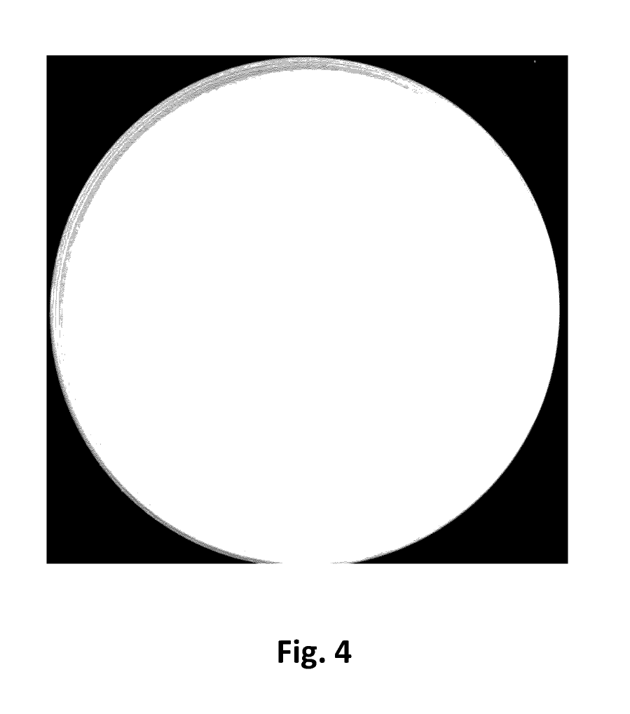

Results in FIG. 4 showed that all recombinant colonies from the combinatorial mutant library showed no parental phenotype (blue color and transparent zone). Over 50% of the colonies had lethal mutations of the two genes (white colonies without transparent zone) and about 35% of the colonies had lethal mutation of Esterase gene only (blue color without transparent zone). The above results demonstrated that the RECODE method with two-step PCR was simple, rapid and can create rich diversity combinatorial mutant library.

(B) Directly Preparation of Double-Stranded Mutant Library without Purifying the Single-Stranded Mutant Library

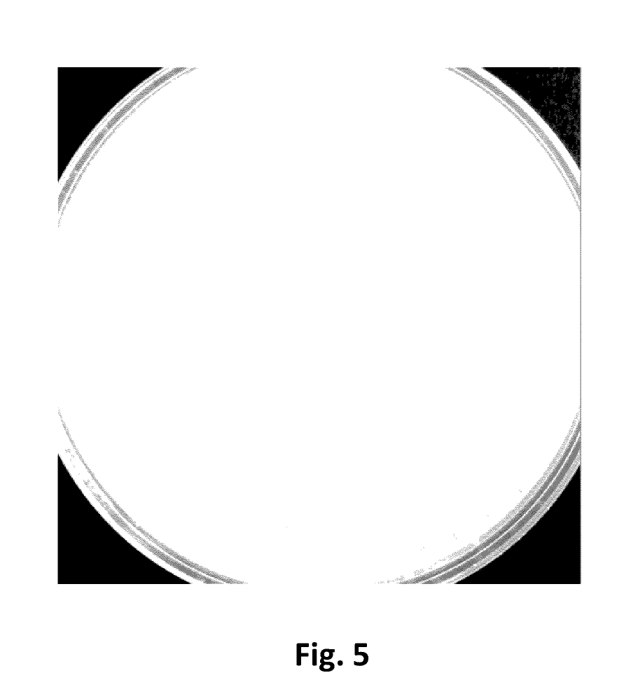

To simplify the operation process, the purification of single-stranded DNA library was omitted by immediately adding excess upstream end primer LZE-F and downstream end primer LZE-R (about 2˜5 folds of the downstream anchor primer) to the products of the first PCR. The mixture system was performed with the following PCR conditions: 3 cycles of 30 s at 94° C., 30 min at 50° C. and 1 min at 68° C.; 5 min at 72° C.; a hold period at 4° C. After that, 0.5 μl Taq DNA polymerase was added to it and hold at 72° C. for 10 min to add an extra Adenine nucleotide to the 3′ end of the products for the subsequent TA-cloning. PCR products were run on 1% agarose gels, and bands of the correct size were excised and purified with a gel extraction kit, according to the manufacturer's protocol. Subsequent cloning steps were the same as described in (A). Results shown in FIG. 5 indicated that double-stranded mutant library constructed using unpurified single mutant library had the similar mutation diversity using purified single mutant library (FIG. 4).

### Example 2

### Construction of Combinatorial Mutant Library by RECODE with One-Step PCR System

Combinatorial lethal mutation of β-galactosidase gene lacZ and Esterase (the same as Example 1) was used as an example to further simplify the RECODE operation process. The three editing primers (JPS-MD/LacZ-P1F, JPS-MD/Est-P2F, JPS-MD/Est-P3F) and the downstream anchor primer JPS-LZE/DTA were mixed at the same concentration and phosphorylated by T4 polynucleotide kinase according to the manufacturer's instruction. Briefly, the reaction was performed for 30 min at 37° C. and the polynucleotide kinase was subsequently heat inactivated for 10 min at 70° C. The preparation of double-stranded mutant library with one-step PCR system was carried out by synchronous PCR cycling reaction of DNA extension, DNA ligation and amplification of the mutant strand DNA.

The 50 μl reaction system contained 5 μl 10× reaction buffer, the upstream anchor primer JPS-LZE/UTA, the phosphorylated downstream anchor primer, appropriate amount of the phosphorylated editing primers (calculated according to the phosphorylation system, 5 pmol of each editing primer was added), end primer LZE-F and LZE-R (about 2 folds of the anchor primer), 1˜5 ng DNA template (the ratio of template to primer was 1:100), 0.5 μl Phusion DNA polymerase (NEB) and 1 μl Ampligase thermostable DNA ligase. The reaction system was mixed and performed with the following PCR conditions: 2 min at 94° C.; 32 cycles of 30 s at 94° C., 1 min at 50° C. and 5 min at 66° C.; 5 min at 72° C.; a hold period at 4° C. The PCR products can be purified and used for further experiments immediately or stored at −20° C.

The PCR products was further treated with Taq DNA polymerase to add an extra Adenine nucleotide, gel extracted and inserted into the pMD19-T vector, and the recombinant pMD19-T vector was transformed into E. coli cells. Similar to Example 1, 50 ng pMD19-T vector was added to equal molar of mutant fragments, mixed and incubated at 16° C. overnight. the whole reaction solution was transformed to competent E. coli cells with normal transformation steps. After cultivated at 16° C. for 1 hour, the transformed cells were cultured on LB plates supplemented with 100 μg·ml⁻¹ Ampicillin, 0.1 mM IPTG, 20 μg·ml⁻¹ X-gal and 1% tributyrin overnight at 37° C.

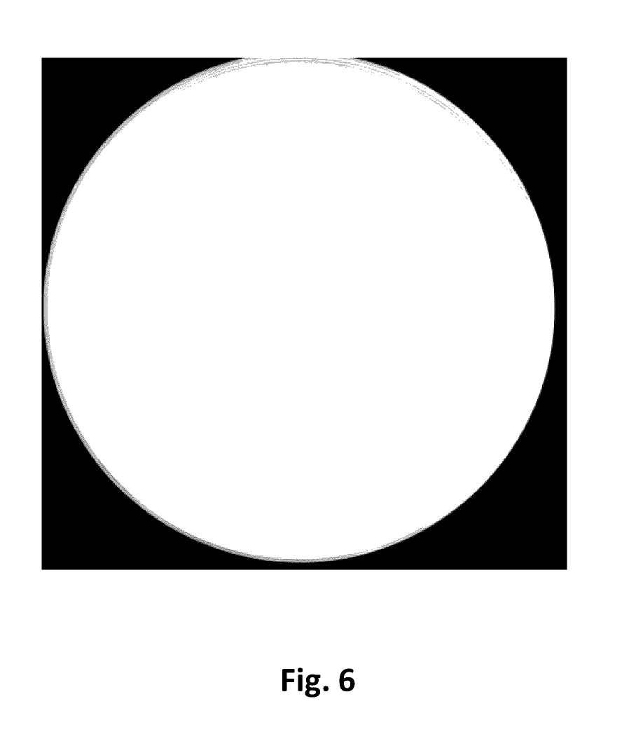

The effect of this one-step method on the mutant library was verified by the colony phenotype in the screening plates (FIG. 6). Although comparatively less target PCR products were obtained in this example, which gave rise to fewer recombinants (concentration of Mg²⁺ or other components in the reaction system affected the generation of double-stranded mutants), the resulting diversity of lethal mutations was similar to that of the two-step PCR.

It can be found that all recombinant colonies from the combinatorial mutant library showed no parental phenotype (blue color and transparent zone). Furthermore, over 60% of the colonies had lethal mutations of the two genes (white colonies without transparent zone) and about 30% of the colonies had lethal mutation of Esterase gene only (blue color without transparent zone). The above results demonstrated that the RECODE method with one-step PCR was more convenient and efficient, and had no negative effect on the diversity of resulting combinatorial mutant library.

### Example 3

### Editing of Constitutive Promoter rpoS Gene from E. Coli In Vitro

The nucleotide sequence of rpoS gene from E. coli was SEQ ID NO:2. Four spacer fragments between the −35 and −10 boxes were combinatorially mutated at the same time and a mutant library was constructed in this example. Four edit primers (Jps/rpoSp-F, Jps/rpoSp4-F, Jps/rpoSp3-F and Jps/rpoSp21-F) were designed according to the target sequence of the promoter, and anchor primers (Jps/rpoSp-HM-F and Jps/rpoSp-HM-R), end primers (rpoSp-F and rpoSp-R) and primers for recombinant vector preparation were designed.

The nucleotide sequences of primers were as follows:

Jps/rpoSp-F:
(SEQ ID No: 12)
TTCCACCGTTGCTGTTGCGTNNNNNNNNNNNNNNNNNTA
TTCTGAGTCTTCGGGT
GAAC
Jps/rpoSp4-F:
(SEQ ID No: 13)
CATAACGACACAATGCTGGTNNNNNNNNNNNNNNAAGTT
AAGGCGGGGCAAAAAATAGC
Jps/rpoSp3-F:
(SEQ ID No: 14)
TAGCACCGGAACCAGTTCAANNNNNNNNNNNNNNNNAAT
TCGTTACAAGGGGAAATCCG
Jps/rpoSp21-F:
(SEQ ID No: 15)
GCAGCGATAAATCGGCGGAACNNNNNNNNNNNNNNNNTG
NTCCGTCAAGGGATCACGGG
Jps/rpoSp-HM-F:
(SEQ ID No: 16)
CACTATAGGGCGAATTGGAGCTCCATACGCGCTGAACGT
TGGTCAG
Jps/rpoSp-HM-R:
(SEQ ID No: 17)
TCAAGGGATCACGGGTAGGAGCCACCGATCCATGGGTAA
GGGAGAAGAAC
rpoSp-F:
(SEQ ID No: 18)
CACTATAGGGCGAATTGGAGCT
rpoSp-R:
(SEQ ID No: 19)
GTTCTTCTCCCTTACCCATG
PBBR2-gfp-F:
(SEQ ID No: 20)
CCATGGGTAAGGGAGAAGAAC
PBBR2-gfp-R:
(SEQ ID No: 21)
CCGGAATTCTTATTTGTATAGTTCATCCATG

The anchor primers (Jps/rpoSp-HM-F and Jps/rpoSp-HM-R) shared the same direction with the editing primers, and they can anneal to the same template strand DNA. In addition, there was anchor sequence (more than 20nt) in the anchor primer, which shares the same sequence with the gap of vector pBBRMCS2-gfp and is used for homologous recombination cloning. The cloning vector was amplified with primers PBBR2-gfp-F and PBBR2-gfp-R. The promoter mutants was inserted to the upstream of gfp (encodes green fluorescent protein(GFP)) for controlling the expression of the gfp and the strength of the promoter mutants can be evaluated by the fluorescence intensity of GFP.

The four editing primers (Jps/rpoSp-F, Jps/rpoSp4-F, Jps/rpoSp3-F and Jps/rpoSp21-F) and the downstream anchor primer Jps/rpoSp-HM-R were mixed at the same concentration and phosphorylated by T4 polynucleotide kinase. Specifically, the phosphorylation reaction systems were prepared according to the manufacturer's instructions. The reaction was performed for 30 min at 37° C. and the polynucleotide kinase was subsequently heat inactivated for 10 min at 70° C.

The preparation of mutant library was carried out by synchronous PCR cycling reaction of DNA extension, DNA ligation and amplification of the mutant strand DNA, using the RECODE method with one-step PCR system described in Example 2.

The RECODE reaction with one-step PCR system was carried out in 25 μl reaction system containing 2.5 μl 10× reaction buffer, the upstream anchor primer Jps/rpoSp-HM-F, the phosphorylated downstream anchor primer, appropriate amount of the phosphorylated editing primers (calculated according to the phosphorylation system, 1˜5 pmol of each editing primer was added), 10˜50 ng DNA template (the ratio of template to primer was 1:100), end primer rpoSp-F and rpoSp-R (about 2 folds of the anchor primer), 0.5 μl Phusion DNA polymerase (NEB) and 1 μl Ampligase thermostable DNA ligase. The reaction system was mixed and performed with the following PCR conditions: 2 min at 94° C.; 32 cycles of 30 s at 94° C., 1 min at 50° C. and 5 min at 66° C.; 5 min at 72° C.; a hold period at 4° C. The PCR products were the promoter mutant library and were run on 1% agarose gels, and bands of the correct size were excised and purified with a gel extraction kit, according to the manufacturer's protocol.

The Gibson assembly method was used for the assembly of the promoter mutant library and vector (Daniel G Gibson. et al. 2009. NATURE METHODS VOL. 6 NO. 5 343-345). The assembly was carried out in 20 μl reaction system containing 8 μl 5× reaction buffer, 1 μl T5 exonuclease (0.2 U·μl⁻¹ Epicentre), 4 μl Taq DNA ligase (40 U·μl⁻¹ New England Biolabs (NEB)) and 0.5 μl Phusion DNA polymerase (2 U·μl⁻¹ NEB). The 5× reaction buffer contained 25% PEG-8000, 500 mM Tris-HCl, 50 min MgCl₂, 50 mM DTT, 1 mM each dNTPs and 5 min NAD, pH 7.5. Specifically, 50 ng linearized vector was added to equal molar of mutant fragments, mixed and incubated at 50° C. for 60 min. The whole reaction solution was the transformed to competent E. coli cells with normal transformation steps. After cultivated at 16° C. for 1 hour, the transformed cells were cultured on LB plates supplemented with 50 μg. Ampicillin at 37° C. overnight.

Screening was performed by high-throughput batch-cultures of the colonies in 96-well microtiter plates. 1000 transformants were randomly picked and placed into microtiter wells and grown in LB broth supplemented with 50 μg·ml⁻¹ kanamycin at 37° C. for 24 h.

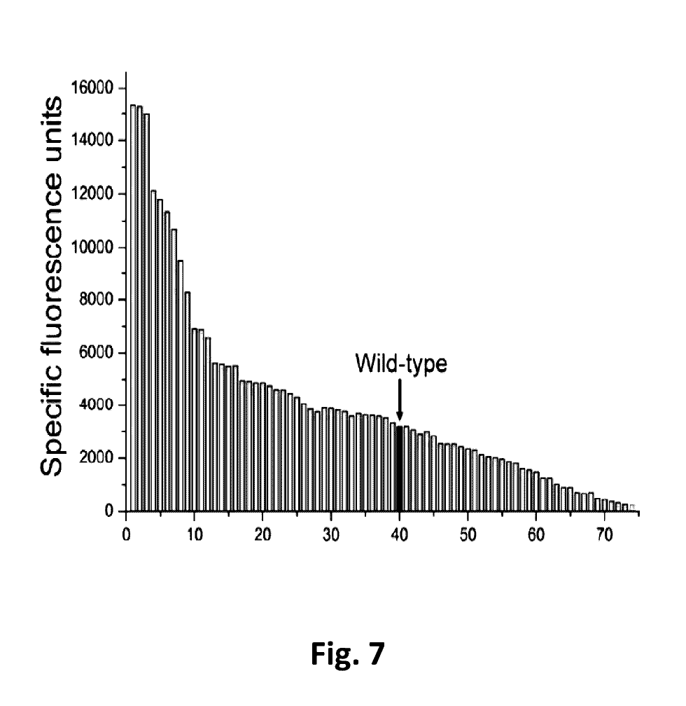

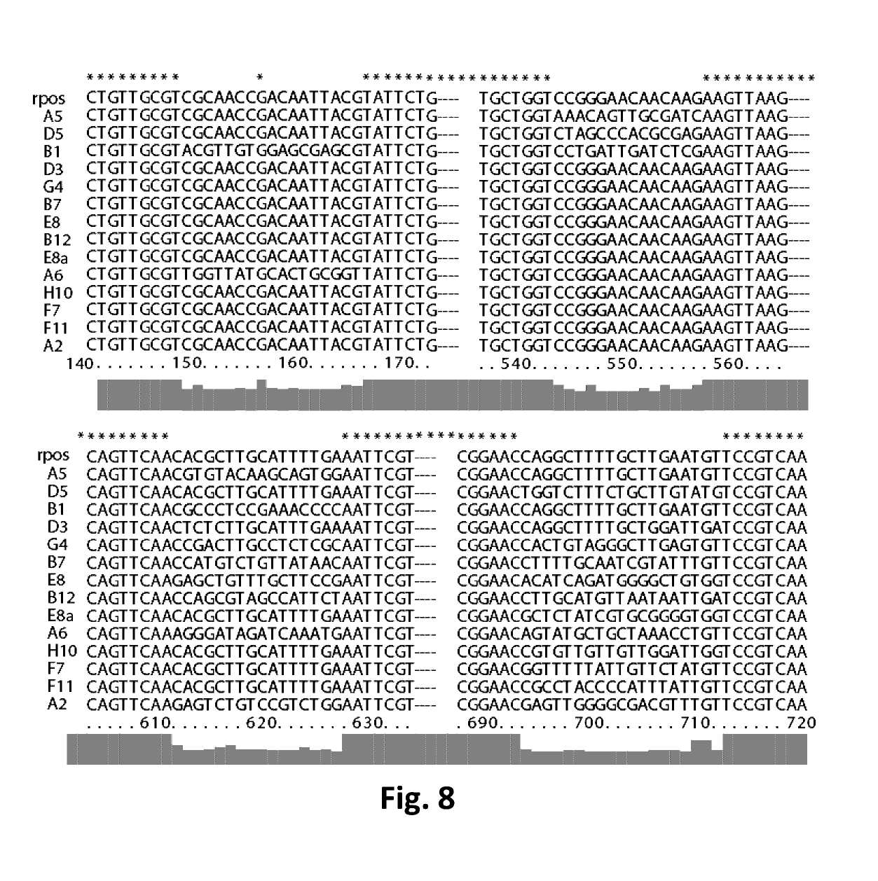

To analyze the activity of the promoter mutant library, cells were collected by centrifugation (3000 rpm, 5 min) The cell pellets were washed three times with sterile water to remove the culture medium and resuspended in appropriate amount of sterile water. The fluorescence intensity and OD₆₀₀ were measured using a microplate reader, and the strength of the promoter mutants was evaluated by the fluorescence intensity value of per OD₆₀₀ cells. As shown in FIG. 7, the variation span of strength of the promoters in the mutant library was 6%-460%, demonstrating the high efficiency of RECODE in combinatorial editing of DNA segments. In addition, DNA sequence analysis (FIG. 8) showed multiple combinatorial mutations with significant differences covering all the four spacer fragments. Furthermore, the number of introduced mutated bases were as many as 10-20, and the introduced mutated bases in different loci were different.

### Example 4

### Editing of Hyaluronidase In Vitro

A leech hyaluronidase gene LHyal was chosen as an example for rapid evolution using RECODE. The nucleotide sequences of LHyal was SEQ ID NO:3. 10 editing primers (JPS/7HF-P1F to JPS/7HF-P10F) were designed to cover the 10 target regions on LHyal gene.

The nucleotide sequences of primers used were as follows:

JPS/7HF-P1F:
(SEQ ID No: 22)
TCTCCGAAAGTTTCCATVNNVNNVNNNNNGATGSGVNNV
NNTTTTCACCGAAAGGGTTG
JPS/7HF-P2F:
(SEQ ID No: 23)
ATCACATCACCGARATTGNNNVNNCTCVNNVNNVNNCTC
TCTCCAGSTTWTTTCCGCGT
JPS/7HF-P3F:
(SEQ ID No: 24)
GTCGGAGGGACGNNNVNNVNNTKGTTAVNNTTTRRCCYC
GATGAAAACAACAAATGGAA
JPS/7HF-P4F:
(SEQ ID No: 25)
GTCAAAYTCRCCAAMKGATCTVNNVNNVNNNTGMTGNTT
NATTTAAACGCTGAAGTCAG
JPS/7HF-P5F:
(SEQ ID No: 26)
AAGGCTATGGAGATNACVNNRRCTGGGAAVNNVNNVNNV
NNCCGGATCATACGTCCGCA
JPS/7HF-P6F:
(SEQ ID No: 27)
CATAAAGTGCTGGAAAAMNATVNNVNNVNNVNNVNNVNN
NCATTANTGGGCCCTGACGT
JPS/7HF-P7F:
(SEQ ID No: 28)
ACGCTTCACCASTACKDSNTTRACGGCMRWNCCKCARMT
RDGAGCACATACCTGGACGC
JPS/7HF-P8F:
(SEQ ID No: 29)
GCACCAAAGATSTTTCGNVVNVVNNTVNNVNNGGTTTTN
TTWSGCTTGACAAACTGGGT
JPS/7HF-P9F:
(SEQ ID No: 30)
AGCCGAATCCAGATTATTGGCTGVNNVNNVNNVNNVNNT
CGTTAGTAGGGMVTACGGTC
JPS/7HF-P10F:
(SEQ ID No: 31)
GAGTGTACGCACAMTGCNCCAAMVNNVNNNCAVNNVNNV
NNCAGAGTCGTTWCTACAAG
JPS/7HF-HM-F:
(SEQ ID No: 32)
GAGGCTGAAGCTTACGTAGAATTCCACCACCACCACCAC
CACATGAAAGAGATCGCGGTGACAATAGACG
JPS/7HF-HM-R:
(SEQ ID No: 33)
TGTAGTCAGCGATGCAAATGTTGAAGCGTGCAAAAAGTA
AGCGGCCGCGAATTAATTCGC
LHyal-F:
(SEQ ID No: 34)
GAGGCTGAAGCTTACGTAGAATTC
LHyal-R:
(SEQ ID No: 35)
GCGAATTAATTCGCGGCCGC

The anchor primers (Jps/7HF-HM-F and Jps/7HF-HM-R) shared the same direction with the editing primers, and they can anneal to the same template strand DNA. In addition, there was 20nt of anchor sequence in the anchor primer, which can be used for homologous recombination cloning. The cloning vector pPIC9K was linearized by digesting with endonuclease EcoRI and NotI. A pair of end primers (LHyal-F and LHyal-R) for amplifying the full-length mutant gene was designed, whose sequences were the same with the anchor sequence or the complementary sequence of the anchor sequence.

The preparation of mutant library was carried out similar to the RECODE method with one-step PCR system described in Example 2. At first, the ten editing primers and the downstream anchor primer Jps/7HF-HM-R were mixed at the same concentration and phosphorylated by T4 polynucleotide kinase, and the polynucleotide kinase was subsequently heat inactivated for 10 min at 70° C. The RECODE reaction with one-step PCR system was carried out in 25 μl reaction system containing 2.5 μl 10× reaction buffer, the upstream anchor primer Jps/7HF-HM-F, the phosphorylated downstream anchor primer Jps/7HF-HM-R, appropriate amount of the phosphorylated editing primers (calculated according to the phosphorylation system, 1˜5 pmol of each editing primer was added), 10˜50 ng DNA template (the ratio of template to primer was 1:100), end primer LHyal-F and LHyal-R (2 folds of the anchor primer), 0.5 μl Phusion DNA polymerase (NEB) and 1 μl Ampligase thermostable DNA ligase. The reaction system was mixed and performed with the following PCR conditions: 2 min at 94° C.; 32 cycles of 30 s at 94° C., 1 min at 50° C. and 5 min at 66° C.; 5 min at 72° C.; a hold period at 4° C. The PCR products were the mutant library.

The PCR products were run on 1% agarose gels, and bands of the correct size were excised and purified with a gel extraction kit according to the manufacturer's protocol. The purified products were assembled with vectors. Specifically, 50 ng linearized pPIC9K was added to equal molar of mutant fragments, mixed and hold at 50° C. for 60 min. The whole reaction solution was transformed into P. pastoris GS 115 with normal transformation steps. After cultivated at 30° C. for 1 hour, the transformed cells were cultured on MD plates supplemented with 2 mg·ml⁻¹G418 at 37° C. for 48 hours.

1000 transformants were randomly picked and transferred into microtiter wells and cultured in buffered methanol minimal yeast medium (BMMY) at 30° C. and 200 rpm for 72 h. In addition, inducer was added to a final concentration of 1% every 24 hours. The BMMY contained 10 g. L⁻¹ yeast extract, 20 g·L⁻¹ peptone, 3 g·L⁻¹ K₂HPO₄, 11.8 g·L⁻¹ KH₂PO₄, 13.4 g. L⁻¹ YNB, 4×10⁻⁴ g·L⁻¹ biotin and 5 mL·L⁻¹ methanol.

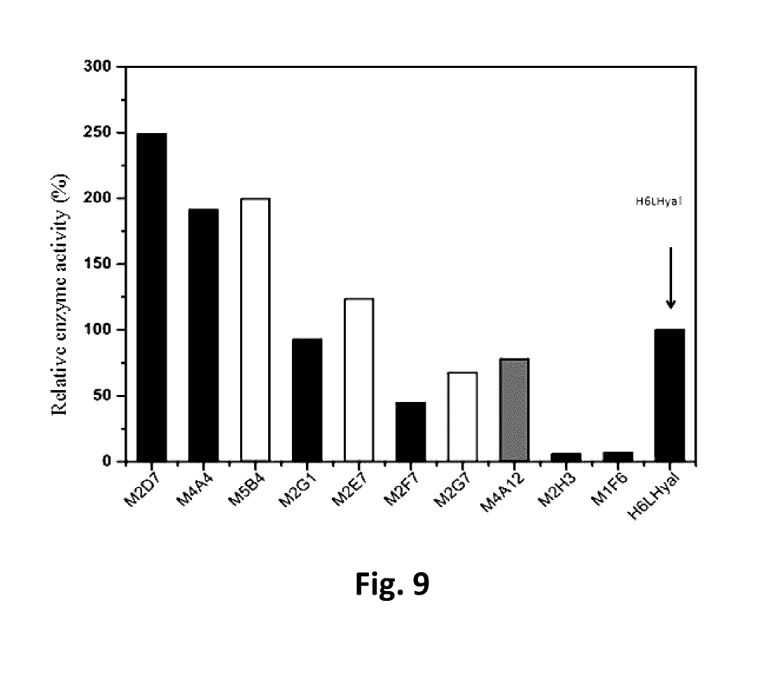

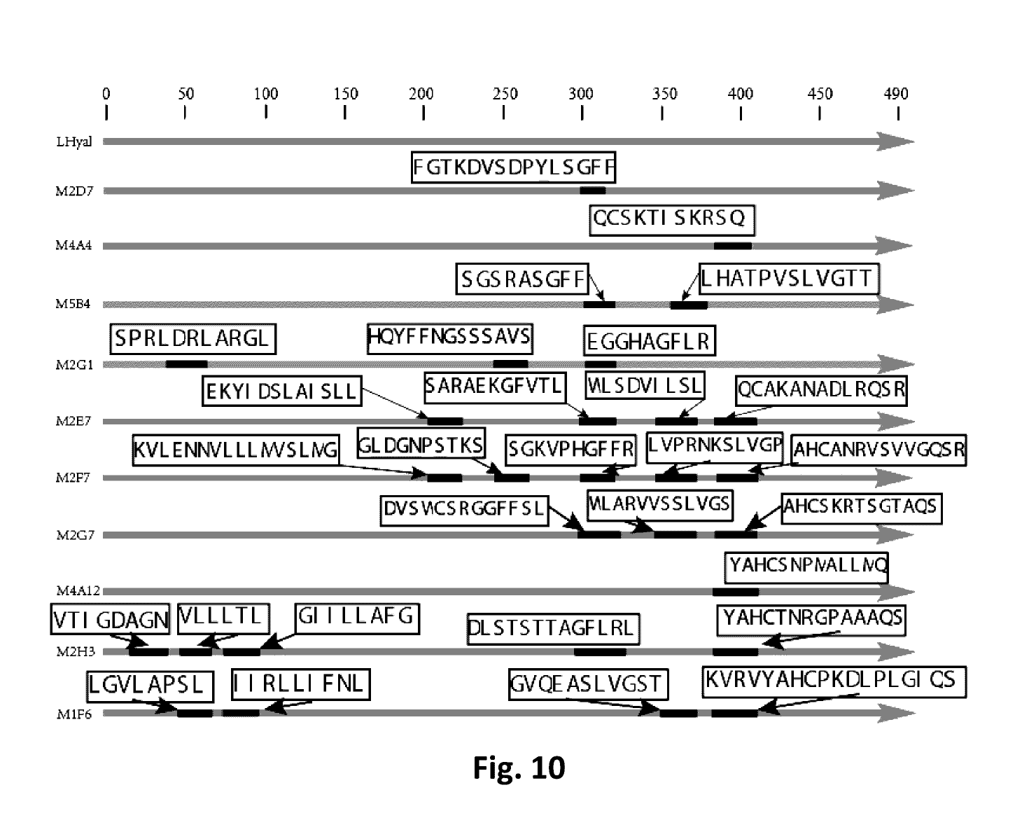

The cultured supernatants of mutants were collected by centrifugation (3500 rpm, 5 min) and diluted for the enzymatic assay in 96-well microtiter plates with an appropriate amount of substrate (hyaluronic acid, 2 mg·mL⁻¹). The enzymatic reaction was incubated at 38° C. for 10 min and terminated by heat inactivation at 95° C. for 2 min, and then examined using the DNS method. The absorbance values were measured using a Microplate Reader at 540 nm and the enzyme activities of the mutants can be calculated according to the standard curve. As shown in FIG. 9, many mutants with significant different activities were successfully created and the mutant M2D7 showed a 2.4-fold increase of the HAase activity. Further DNA sequencing analysis of these mutants showed that the random combinations of mutation regions exhibited rich diversity and almost all the pre-designed mutation regions were covered with random combinations. In addition, the number of combination positions introduced into the mutation regions ranged from one to five, and more than 30 mutated amino acid residues were introduced into the mutants M2F7 and M2H3 (FIG. 10). This example demonstrates the powerful capacity of the RECODE method in directed enzyme evolution.

### Example 5

### Editing of Synthetic Pathway of 5-Aminolevulinic Acid In Vitro

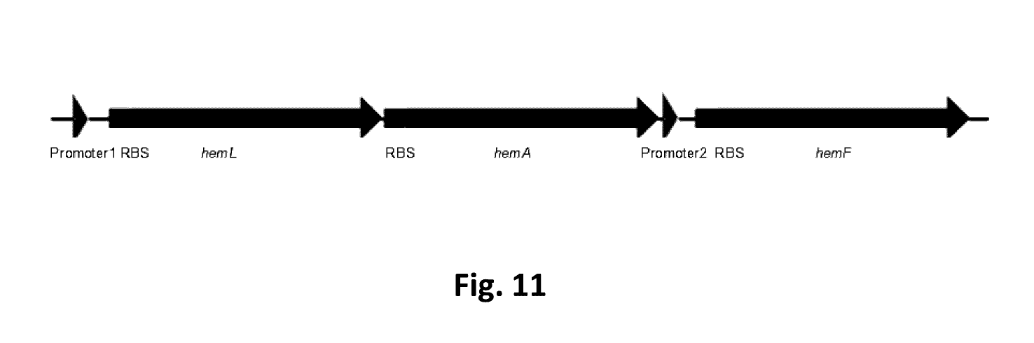

On the basis of the established simple RECODE method, a novel approach of combinatorial engineering of regulatory elements (such as promoters and RBS) and pathway enzymes was proposed to construct an optimized synthetic pathway towards the target compound. As an example, for combinatorial evolution of the 5-aminolevulinic acid (ALA) pathway, the three pathway genes (hemA, hemL and hemF) were simultaneously engineered at the transcriptional and protein levels with a starting nucleotide sequence (SEQ ID NO:3) shown as FIG. 11, which was inserted into plasmid pRSFduet-1. Fourteen editing primers (from JPS/LAF-P1 to JPS/LAF-P14F) were designed and the target regions were the three RBS of the three genes, the spacer sequences between −35 and −10 boxes of the hemF promoter and suitable internal sites of protein encoding region of the three genes.

The nucleotide sequences of primers used were as follows:

JPS/LAF-P1F:
(SEQ ID No: 36)
AACTTTAATAAGGAGGGATCCATAAAAGGRRRDDDDTATATGAGTAA
GTCTGAAAATCT
JPS/LAF-P2F:
(SEQ ID No: 37)
CGTGGTTTAAGCTTTGGTNCANCANNCVNNVNNVNNNTGNAAATGGC
GCAACTGGTGA
JPS/LAF-P3F:
(SEQ ID No: 38)
CGCCTGGCCCGTGGTTTTNCCNNTVNNVNNANNNNTNTTAAATTTGA
AGGGTGTTACC
JPS/LAF-P4F:
(SEQ ID No: 39)
ACTTATAATGATCTGGCTNCTVTAVNNVNNNCANNTVNGCAATACCC
GCAAGAGATTGC
JPS/LAF-P5F:
(SEQ ID No: 40)
GGTGGTCGTCGTGATGTAATGNATVNNVNNVNNVNNNCGNGTCCGGT
CTATCAGGCGG
JPS/LAF-P6F:
(SEQ ID No: 41)
GGTGTTTGCGAAGTTGTAADDRRRRRDDDDDATGNNCVNNVNGCTTT
TAGCGCTCGGTA
JPS/LAF-P7F:
(SEQ ID No: 42)
AATGACGCCGTCAGCCACNTGNTGVNNVNNVNNNGCNGTCTGGATTC
ACTGGTGCTGGG
JPS/LAF-P8F:
(SEQ ID No: 43)
AGCGCCGTCTCCGTCGCGNTTNCCVNNNNTVNNVNNGCCCGCCAAAT
CTTTGAATCGCT
JPS/LAF-P9F:
(SEQ ID No: 44)
ATTATCGCCAACCGAACCNGCVAGVNNVNNVAANCCCTGGCGGATGA
GGTAGGCGCCG
JPS/LAF-P10F:
(SEQ ID No: 45)
GCATTAAAAAGCCGTCGTNACVNGVNNNTGVNNVNNGTGGATATCGC
CGTACCGCGCG
JPS/LAF-P11F:
(SEQ ID No: 46)
CCGCATAATCGAAATTAATANNNNNNNNTATAGGGGAATTGTGAGCG
GATAAC
JPS/LAF-P12F:
(SEQ ID No: 47)
GTATATTAGTTAAGTATAAGRRRRRRDDDDDDATGAAACCCGACGCA
CACCAGG
JPS/LAF-P13F:
(SEQ ID No: 48)
CATCGCCCGGAACTTGCCNGGVNNVNNNNCGAGGCGATGGGCGTTTC
ACTG
JPS/LAF-P14F:
(SEQ ID No: 49)
TTCTATGGTTTTGAAGAAGATNCTNNTNNNNNGNATCGCACCGCCCG
TGACCTGTGCC
JPS/LAF-P15F:
(SEQ ID No: 50)
GGCGTTAAGTGAGTTTATTAAGGTCAGGGATTGGGTGTAAGCACTTC
GTGGCCGAGCTCG
JPS/LAF-ELF:
(SEQ ID No: 51)
TGTTTAACTTTAATAAGGAGGGATCC
JPS/LAF-ELR:
(SEQ ID No: 52)
CGAGCTCGGCCACGAAGTGC
PRSFDUET-F:
(SEQ ID No: 53)
GCACTTCGTGGCCGAGCTCGAGTCTGGTAAAGAAACCGCTGC
PRSFDUET-R:
(SEQ ID No: 54)
CTCCTTATTAAAGTTAAACAAAATTATTTCTACAG

The preparation of mutant library for metabolic pathway evolution was carried out by RECODE method with one-step PCR system described in Example 2. Editing primer JPS/LAF-P1F had the function of anchor primer (having an anchor sequence), JPS/LAF-P15F was another editing primer with an anchor sequence, JPS/LAF-ELF and JPS/LAF-ELR were end primers, and PRSFDUET-F/PRSFDUET-R were primers for amplification of recombinant plasmid.

The RECODE reaction with one-step PCR system was carried out in 25 μl reaction system containing 2.5 μl 10× reaction buffer, the upstream anchor primer JPS/LAF-P1F, the phosphorylated downstream anchor primer JPS/LAF-P15F, appropriate amount of the phosphorylated editing primers (calculated according to the phosphorylation system, 1˜5 pmol of each editing primer was added), 10˜50 ng DNA template (the ratio of template to primer was 1:100), end primer JPS/LAF-ELF and JPS/LAF-ELR (2 folds of the anchor primer each), 0.5 μl Phusion DNA polymerase and 1 μl Ampligase. The reaction system was mixed and performed with the following PCR conditions: 2 min at 94° C.; 32 cycles of 30 s at 94° C., 1 min at 50° C. and 5 min at 66° C.; 5 min at 72° C.; a hold period at 4° C. PCR products were the mutant library of engineered ALA pathway and were run on 1% agarose gels, and bands of the correct size were excised and purified with a gel extraction kit, according to the manufacturer's protocol.

The ALA pathway library assembled into pRSFDuet-1 vector was transformed into E. coli BL21 (DE3) competent cells and cultured on LB plates supplemented with 50 μg·ml⁻¹ kanamycin overnight at 37° C. Screening was performed by high-throughput batch-cultures in 96-well microtiter plates to analyze the ALA yields from the mutants. 2400 transformants were randomly picked and placed into microtiter wells and cultured in mineral basal medium at 37° C. 200 rpm for 36 hours. Culture supernatants of the mutants were collected and diluted for detection of the ALA production. The mineral basal medium (pH 7.0) contained 20.0 g. L⁻¹ glucose, 2.0 g. L⁻¹ yeast extract, 16.0 g. L⁻¹ (NH₄)₂SO₄, 3.0 g. L⁻¹ KH₂PO₄, 16.0 g. L⁻¹ Na₂HPO₄.12H₂O, 1.0 g. L⁻¹ MgSO₄.7H₂O, 0.01 g. L⁻¹ MnSO₄.H₂O, 50 μg·ml⁻¹ kanamycin and 0.1 mM IPTG.

Detection of the ALA production was carried out as follows: dilute the sample to 2 mL, add 1 mL acetate buffers and 0.5 mL acetylacetonate; boil for 15 min and cool to room temperature; add 2 mL reaction solution and 2 mL modified Ehrlich's reagents to a new tube; react for 20 min and detect the product at 554 nm using a spectrophotometer. When 96-well microtiter plates were used for detection, the amount of reagents was reduced correspondingly.

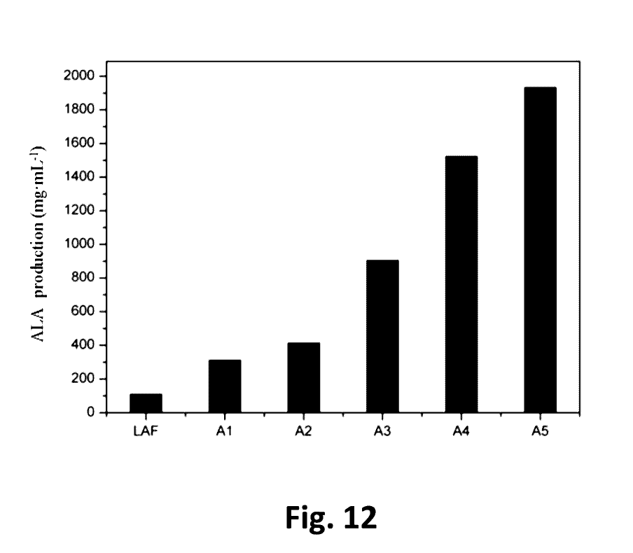

As shown in FIG. 12, the ALA production of the parental strain was only 110 mg·L⁻¹, while mutant strains with significant enhanced ALA production were screened from the ALA pathway library. The recombinant BL21(DE3) strain A5 accumulated ALA to the highest titer 1930 mg·L⁻¹, which was 17.5-fold of that of the parental strain. The results demonstrated the high efficiency of this simple approach in pathway engineering.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

What is claimed is:

1. A method of rapid directed DNA evolution using a combinatorial DNA mutant library, comprising the steps of:
a) preparing a single-stranded DNA mutant library, wherein editing primers with mutated nucleotide(s) that are targeted to different regions of the same strand of a parent DNA are used as internal primers in a PCR amplification to introduce combinatorial mutations into said parent DNA, and wherein single-stranded DNAs with said combinatorial mutations constitute said single-stranded DNA mutant library;
b) preparing a double-stranded DNA mutant library, wherein said single-stranded DNA mutant library is used as a template to generate said double-stranded DNA mutant library; and
c) assembling said double-stranded DNA mutant library into expression vectors and screening for mutants with desired properties.

2. The method of claim 1, wherein said editing primer comprises at least one mutagenic nucleotide, and all of said editing primers have the same direction and anneal to the same strand of said parent DNA, wherein said editing primers are used as internal primers to generate mutated DNA fragments using a DNA polymerase, wherein said mutated DNA fragments are ligated together to form a full-length mutated DNA strand using a DNA ligase.

3. The method of claim 2, wherein said mutagenic nucleotide(s) is placed in the middle of said editing primer and, optionally, can be highly degenerated.

4. The method of claim 1, wherein said parent DNA is a regulatory DNA for regulating gene expression, a protein encoding gene, or a combination of both.

5. The method of claim 1, wherein said parent DNA is a plasmid, a genomic sequence, a double-stranded DNA, or a single-stranded DNA.

6. The method of claim 2, wherein upstream and downstream anchor primers are added to the PCR system for generating said single-stranded DNA mutants, wherein said upstream and downstream anchor primers have the same direction as said editing primers and anneal to the upstream and downstream location, respectively, of all the regions targeted by said editing primers, wherein said upstream and downstream anchor primers comprise sequences complimentary to said parent DNA and an anchor sequence at 5′ and 3′ ends of said upstream and downstream anchor primers, respectively, wherein said anchor sequence is a unique sequence specific for said single-stranded DNA mutants and can be used for designing end primers to specifically amplify the full-length sequence of said single-stranded DNA mutants.

7. The method of claim 6, wherein said upstream and downstream anchor primers comprise mutagenic nucleotides.

8. The method of claim 6, wherein a pair of end primers based on said anchor sequence are used to amplify said full-length single-stranded mutant DNA and convert said single-stranded mutant DNA to said double-stranded mutant DNA.

9. The method of claim 8, wherein said single-stranded DNA mutant library and double-stranded DNA mutant library are constructed in the same PCR system, wherein said PCR system comprises said parent DNA, said editing primers, said upstream and downstream anchor primers, said end primer pairs, said DNA polymerase and said DNA ligase, and wherein said single-stranded DNA mutant, once produced, is used a template to generate said double-stranded DNA mutant.

10. The method of claim 1, wherein said single-stranded mutant library, either purified or non-purified, is used as a template to generate said double-stranded mutant library.

11. The method of claim 1, wherein said double-stranded DNA mutant library is assembled into expression vectors by in vitro recombinant techniques.

12. The method of claim 11, wherein said in vitro recombinant technique is selected from fusion PCR, Gibson assembly, T4 DNA polymerase-mediated recombinant techniques and in vivo yeast assembly techniques.

13. The method of claim 2, wherein said DNA ligase is a thermostable DNA ligase.

14. The method of claim 13, wherein said thermostable DNA ligase is selected from Taq DNA ligase, 9N DNA ligase and Ampligase.

15. The method of claim 1, wherein said DNA polymerase is a thermostable, high-fidelity DNA polymerase, preferably without 5′-3′ exonuclease activity.

16. The method of claim 15, wherein said DNA polymerase is Phusion DNA polymerase.
